(19) United States
(12) Patent Application Publication
(10) Pub. No.: US 2025/0281312 A1
(43) Pub. Date: Sep. 11, 2025

(54) STENT TISSUE COUPLING

(71) Applicant: EDWARDS LIFESCIENCES CORPORATION, Irvine, CA (US)

(72) Inventors: Roei Mayo, Irvine, CA (US); Emil Karapetian, Huntington Beach, CA (US)

(73) Assignee: EDWARDS LIFESCIENCES CORPORATION, Irvine, CA (US)

(21) Appl. No.: 19/215,145

(22) Filed: May 21, 2025

Related U.S. Application Data
(63) Continuation of application No. PCT/US2023/084268, filed on Dec. 15, 2023.
(60) Provisional application No. 63/476,129, filed on Dec. 19, 2022.
(60) Provisional application No. 63/582,827, filed on Sep. 14, 2023.

(51) Int. Cl. A61F 2/82; A61F 2/95

(52) U.S. Cl. CPC A61F 2/82; A61F 2/95; A61F 2230/0006; A61F 2230/0008; A61F 2230/001; A61F 2230/0069

(57) ABSTRACT

A method of coupling a stent to a blood vessel involves deploying a stent in a target blood vessel segment, the stent having a non-circular biased shape, forcing the stent to a more-circular shape compared to the non-circular biased shape using a tie coupled across an inner diameter of the stent in a tensioned state, maintaining the tie in the tensioned state for a period of time sufficient to allow tissue overgrowth to couple the stent to a wall of the target blood vessel segment, and de-tensioning the tie to allow the stent to reshape the blood vessel segment to a non-circular shape.

### RELATED APPLICATIONS

This application is a continuation of International Patent Application No. PCT/US23/84268, filed Dec. 15, 2023, which claims the benefit of U.S. Provisional Patent Application Ser. No. 63/476,129, filed on Dec. 19, 2022, and U.S. Provisional Patent Application Ser. No. 63/582,827, filed on Sep. 14, 2023, the complete disclosures of which are hereby incorporated by reference in their entireties.

### BACKGROUND

The present disclosure generally relates to the field of medical implant devices. Insufficient or reduced compliance in certain blood vessels, including arteries such as the aorta, can result in reduced perfusion, cardiac output, and other health complications. Restoring compliance and/or otherwise controlling flow in such blood vessels can improve patient outcomes.

### SUMMARY

Described herein are devices, methods, and systems that facilitate coupling of a non-circular stent with a blood vessel through tissue ingrowth. Devices associated with the various examples of the present disclosure can include one or more support structures configured to force a stent, or at least a portion thereof, that has a non-circular biased cross-sectional shape, into a more-circular shape. Stent-circularizing supports of the present disclosure can take the form of stents, ties/tethers, or other forms. The support structure(s), by forcing the more-circular shape of the stent or stent portion, facilitate(s) contact between the stent and the blood vessel wall of a blood vessel in which the stent is implanted for a period of time sufficient to allow for endothelialization, or other tissue overgrowth, of the stent. Once sufficient tissue overgrowth of the stent has occurred, removal of the support(s) can allow for the stent to assume a biased non-circular shape thereof, wherein such transition to a non-circular shape can cause reshaping of the blood vessel, which is physically coupled to the sent by means of tissue integration therewith. Removal of the stent support(s) can be achieved through dissolution of the support(s), which may comprise biodegradable material configured to dissolve after a period of time sufficient to accommodate the tissue overgrowth of the stent. With the tissue overgrowth of the stent, the stent may be used to reshape the blood vessel in at least certain periods/phases of the cardiac cycle. Such reshaping of the blood vessel, forced by the cyclical reshaping of the stent (or segment thereof), can promote compliant blood flow/pressure leveling by reducing systolic pressure/flow and/or increasing diastolic pressure/flow.

In some aspects, the techniques described herein relate to an implant device including: a frame having a biased non-circular axial cross-sectional shape; and a covering attached to the frame and enclosing a first portion of the frame, wherein a second portion of the frame is not enclosed by the covering.

In some aspects, the techniques described herein relate to an implant device, further including a tie configured to be disposed within an inner channel of the frame and to force the frame to a shape that is more circular than the biased non-circular shape.

In some aspects, the techniques described herein relate to an implant device, wherein the tie is biodegradable.

In some aspects, the techniques described herein relate to an implant device or claim 3, wherein the tie includes biodegradable material.

In some aspects, the techniques described herein relate to an implant device, wherein the frame and the tie are radially compressible.

In some aspects, the techniques described herein relate to an implant device, wherein the frame and the tie are configured to be transported together in a delivery system in which the frame is radially compressed and the tie is radially compressed and disposed within the radially compressed frame.

In some aspects, the techniques described herein relate to an implant device, wherein the biased non-circular shape of the frame has a major-axis dimension and a minor-axis dimension, the major-axis dimension being greater than the minor-axis dimension.

In some aspects, the techniques described herein relate to an implant device, wherein the tie has a diameter that is greater than the minor-axis dimension of the frame and smaller than the major-axis dimension of the frame.

In some aspects, the techniques described herein relate to an implant device or claim 8, wherein the biased non-circular shape is an oval shape.

In some aspects, the techniques described herein relate to an implant device, wherein the biased non-circular shape is a peanut shape.

In some aspects, the techniques described herein relate to an implant device, wherein the frame includes generally circular bulbous portions on opposing ends of the frame, and wherein the frame includes a midsection between the bulbous portions.

In some aspects, the techniques described herein relate to an implant device, wherein the covering encloses the midsection.

In some aspects, the techniques described herein relate to an implant device or claim 12, wherein the covering encloses at least one of the bulbous portions.

In some aspects, the techniques described herein relate to an implant device, wherein the covering does not enclose at least one of the bulbous portions.

In some aspects, the techniques described herein relate to an implant device, wherein the midsection includes inwardly-deflected side walls.

In some aspects, the techniques described herein relate to an implant device, wherein at least one of the bulbous portions is configured to be aligned with one or more branching blood vessels.

In some aspects, the techniques described herein relate to an implant device, wherein the covering is biodegradable.

In some aspects, the techniques described herein relate to a method of coupling a stent to a blood vessel, the method including: deploying a stent in a target blood vessel segment, the stent having a non-circular relaxed shape and including a covering enclosing at least a portion of the stent; and aligning a first end of the stent with one or more branching blood vessels that branch from the target blood vessel segment, wherein the first end is not enclosed by the covering.

In some aspects, the techniques described herein relate to a method, further including: forcing the stent to a more-circular shape compared to the non-circular relaxed shape using a tie disposed in an inner channel of the stent; maintaining the tie within the stent for a period of time sufficient to allow tissue overgrowth to couple the stent to a wall of the target blood vessel segment; and removing the tie to allow the stent to reshape the target blood vessel segment to a non-circular shape.

In some aspects, the techniques described herein relate to a method or claim 19, wherein the covering is configured to cause in-growth of tissue.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Any of the example methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loudspeakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates example cardiac and vascular anatomy of a patient.

FIG. 2A shows an example healthy aorta.

FIGS. 2B and 2C show side and axial cross-sectional views, respectively, of the healthy aorta of FIG. 2A experiencing compliant expansion and contraction over a cardiac cycle.

FIG. 3A shows an example stiff aorta.

FIGS. 3B and 3C show side and axial cross-sectional views, respectively, of the stiff aorta of FIG. 3A experiencing compromised expansion and contraction over a cardiac cycle.

FIGS. 4-1 and 4-2 show a blood vessel in circular and non-circular shapes, respectively.

FIGS. 5A and 5B show perspective and axial views, respectively, of a non-circular stent disposed in a blood vessel.

FIGS. 6A and 6B show perspective and axial views, respectively, of a non-circular stent disposed in a blood vessel in accordance with one or more examples.

FIGS. 7-1 and 7-2 show oval and peanut-shaped stents, respectively, disposed in a blood vessel in accordance with one or more examples.

FIGS. 8A and SB show a stent-type inner circularizing support disposed within a stent to force a circular shape thereof in accordance with one or more examples.

FIG. 9 shows a non-circular stent coupled to blood vessel wall(s) via tissue overgrowth facilitated by a circularizing support in accordance with one or more examples.

FIGS. 10A, 10B, and 10C illustrate a flow diagram for a process for coupling a non-circular stent to a blood vessel using a circularizing stent in accordance with one or more examples.

FIGS. 11-1, 11-2A, 11-2B, 11-3, 11-4A, and 11-4B provide images of the compliance-enhancing implant device and certain anatomy corresponding to operations of the process of FIGS. 10A, 10B, and 10C according to one or more examples.

FIGS. 12A and 12B show a tie-type circularizing support secured to a stent to force a circular shape thereof in accordance with one or more examples.

FIG. 13 shows a non-circular stent coupled to blood vessel wall(s) via tissue overgrowth facilitated by a circularizing support in accordance with one or more examples.

FIGS. 14A and 14B illustrate a flow diagram for a process for coupling a non-circular stent to a blood vessel using a circularizing stent in accordance with one or more examples.

FIGS. 15-1A, 15-1B, and 15-2 provide images of the compliance-enhancing implant device and certain anatomy corresponding to operations of the process of FIGS. 14A and 14B according to one or more examples.

FIG. 16 shows an axial view of a non-circular stent having inwardly-deflected sidewalls in accordance with one or more examples.

FIG. 17 shows an axial view of a non-circular stent having inwardly-deflected sidewalls in accordance with one or more examples.

FIG. 18 shows an axial view of a peanut-shaped stent deployed within a blood vessel in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Vascular Anatomy and Compliance

Certain examples are disclosed herein in the context of vascular implant devices, and in particular, stent-shaping implant devices implanted in the aorta. However, although certain principles disclosed herein may be particularly applicable to the anatomy of the aorta, it should be understood that stent-shaping implant devices in accordance with the present disclosure, and/or inventive stent devices having circularizing support features coupled to and/or otherwise associated therewith, may be implanted in, or configured for implantation in, any suitable or desirable blood vessels or other anatomy, such as the inferior vena cava. The terms “associated” and “associated with” are used herein according to their broad and ordinary meanings. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart.

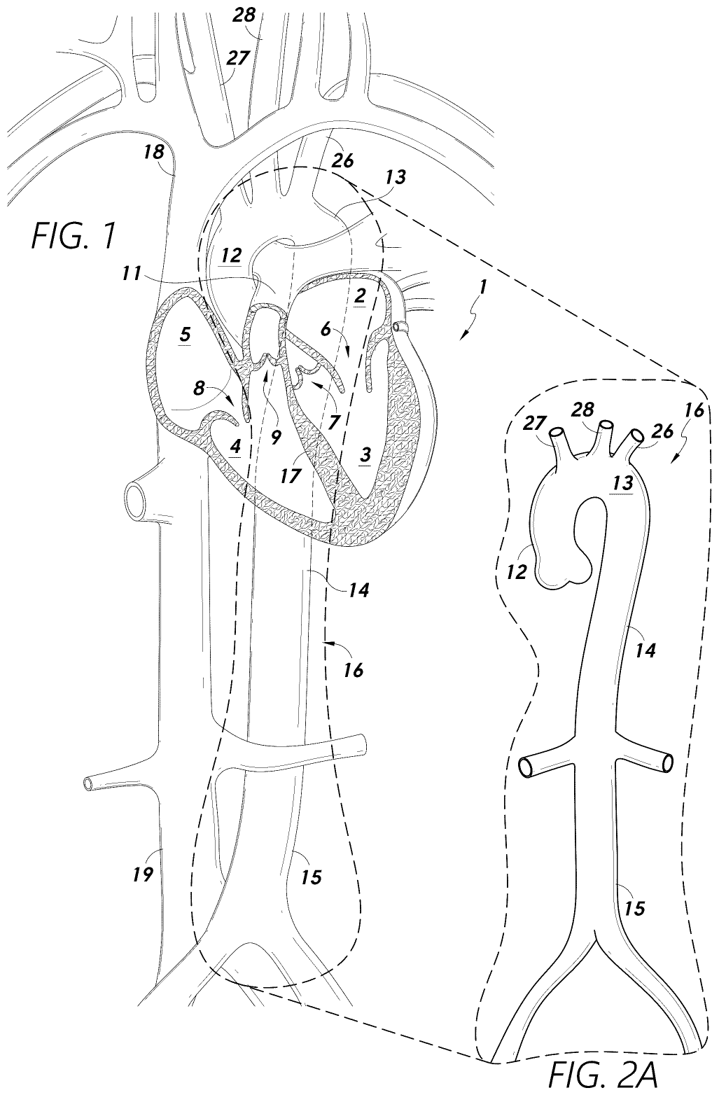

FIG. 1 illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

The atrioventricular (mitral and tricuspid) heart valves generally are coupled to a collection of chordae tendineae and papillary muscles (not shown for visual clarity) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle 17, referred to as the septum, separates the left 2 and right 5 atria and the left 3 and right 4 ventricles.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior 19 and superior 18 venae cavae, carry blood back to the heart.

The aorta 16 is a compliant arterial blood vessel that buffers and conducts pulsatile left ventricular output and contributes the largest component of total compliance of the arterial tree. The aorta 16 includes the ascending aorta 12, which begins at the opening of the aortic valve 7 in the left ventricle of the heart. The ascending aorta 12 and pulmonary trunk 11 twist around each other, causing the aorta 12 to start out posterior to the pulmonary trunk 11, but end by twisting to its right and anterior side. Among the various segments of the aorta 16, the ascending aorta 12 is relatively more frequently affected by aneurysms and dissections, often requiring open heart surgery to be repaired. The transition from ascending aorta 12 to aortic arch 13 is at the pericardial reflection on the aorta. At the root of the ascending aorta 12, the lumen has three small pockets between the cusps of the aortic valve and the wall of the aorta, which are called the aortic sinuses or the sinuses of Valsalva. The left aortic sinus contains the origin of the left coronary artery and the right aortic sinus likewise gives rise to the right coronary artery. Together, these two arteries supply the heart.

As mentioned above, the aorta 16 is coupled to the heart 1 via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

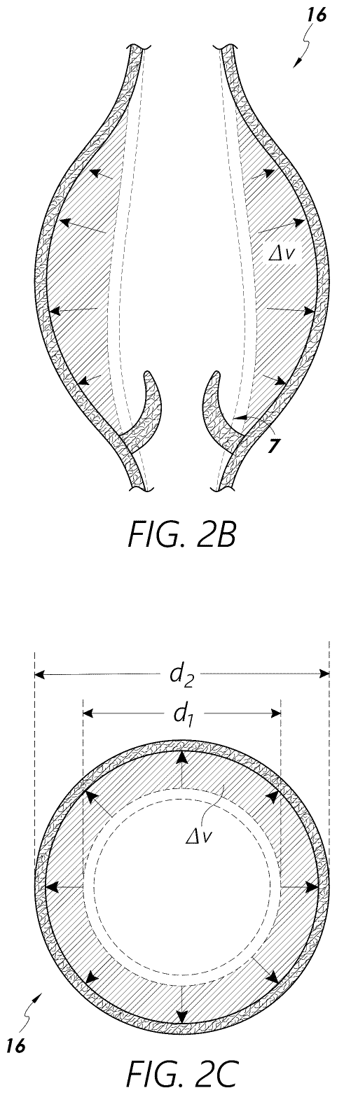

FIG. 2A shows an example healthy aorta 16. FIGS. 2B and 2C show side and axial cross-sectional views, respectively, of the healthy aorta 16 of FIG. 2A experiencing compliant expansion and contraction over a cardiac cycle.

As referenced above, the systolic phase of the cardiac cycle is associated with the pumping phase of the left ventricle, while the diastolic phase of the cardiac cycle is associated with the resting or filling phase of the left ventricle. As shown in FIGS. 2A and 2B, with proper arterial compliance, an increase in volume Δv will generally occur in an artery when the pressure in the artery is increased from diastole to systole. As blood is pumped into the aorta 16 through the aortic valve 7, the pressure in the aorta increases and the diameter of at least a portion thereof expands. A first portion of the blood entering the aorta 16 during systole may pass through the artery during the systolic phase, while a second portion (e.g., approximately half of the total blood volume) may be stored in the expanded volume Δv caused by compliant stretching of the blood vessel 16 from a non-expanded diameter d₁ to an expanded diameter d₂, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where Δv is the change in volume (e.g., in mL) of the blood vessel, and Δp is the pulse pressure from systole to diastole (e.g., in mmHg):

$$C = \frac{Δ v}{Δ p} \qquad ( 1 )$$

In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

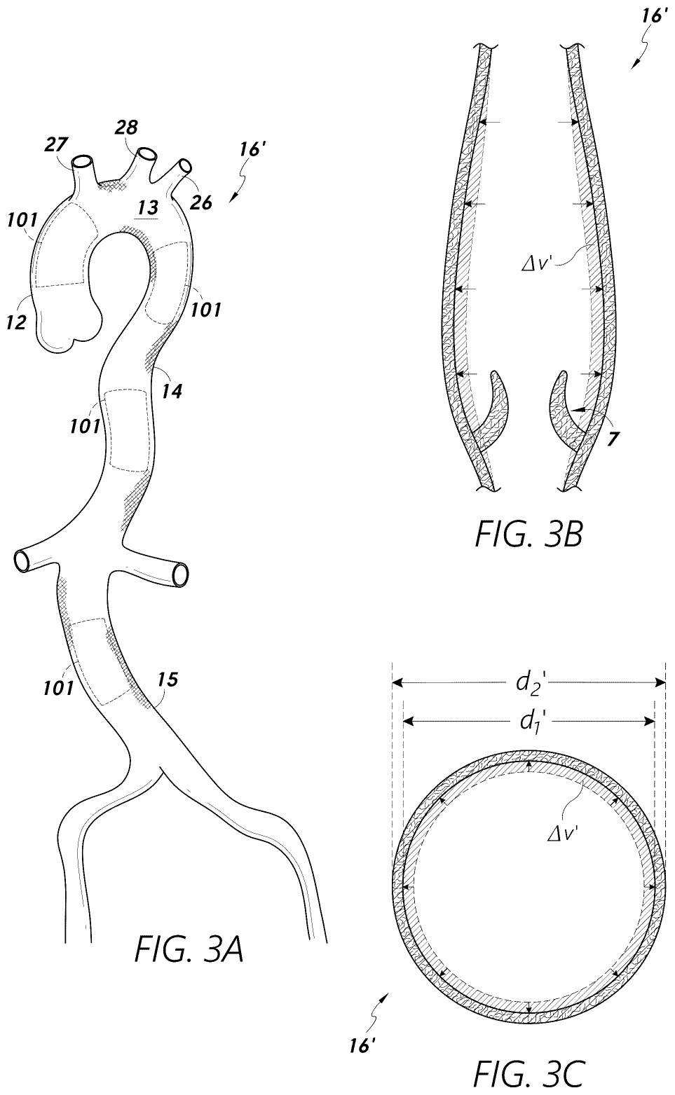

FIG. 3A shows an example stiff aorta 16′. FIGS. 3B and 3C show side and axial cross-sectional views, respectively, of the stiff aorta 16′ of FIG. 3A experiencing compromised expansion and contraction over a cardiac cycle.

As shown in FIG. 3A, the aorta tends to change in shape as a function of age, resulting in a higher degree of curvature and/or tortuosity over time. As the vasculature of a subject becomes less elastic, arterial blood pressure (e.g., left-ventricular afterload) becomes more pulsatile, which can have a deleterious effect. For example, undesirably pulsatile arterial blood flow, such as the thickening of the left ventricle muscle and/or diastolic heart failure. Stiffness in the aorta and/or other blood vessel(s) can occur due to an increase in collagen content and/or a corresponding decrease in elastin.

With the walls of the blood vessel 16′ being resistant to stretching due to the stiffness thereof, the expansion of the blood vessel diameter from the non-expanded diameter d₁′ to the expanded diameter d₂′ may be limited/reduced compared to the expansion of diameter of a healthy blood vessel. Although FIGS. 3B and 3C show a small amount of expansion and volume change Δv′ experienced by the blood vessel 16′, in some cases, a blood vessel may be sufficiently stiff that substantially no vessel expansion takes place in systole.

Generally, the majority of aortic compliance is provided in the ascending aorta 12 with respect to healthy anatomy. Furthermore, calcification frequently occurs in the area of the ascending aorta 12, near the aortic arch 13 and the great vessels emanating therefrom. Such anatomical areas can experience relatively higher stresses due to the geometry, elasticity, and flow dynamics associated therewith. Therefore, implantation/deployment of compliance-enhancing stent devices secured to blood vessel walls using circularizing support devices of the present disclosure can advantageously be in the ascending aorta 12 in some cases. While relatively less calcification tends to occur in the descending 14 and abdominal 15 aorta, implant devices of the present disclosure can advantageously be implanted/deployed in such areas as well for the purpose of increasing compliance in the aortic system. Examples of the present disclosure provide support structures/devices, which may be implanted/secured within non-circular stent devices in one or more locations in a compromised aorta and/or other vessel(s). For example, FIG. 3A shows example positions of stents 101 secured to blood vessel walls using features/aspects disclosed herein, such stents and/or circularizing support structures/devices being implanted/disposed in various potential areas of an aorta 16′.

### Blood-Vessel-Reshaping Stent Implants

The present disclosure relates to systems, devices, and methods for adding-back and/or increasing compliance in the aorta or other arterial (or venous) blood vessel(s) to provide improved perfusion of the heart muscle and/or other organ(s) of the body. Examples of the present disclosure can include support devices configured to reshape/circularize stents that, when implanted and freed from the reshaping/circularizing support(s) (e.g., support(s) dissolved or otherwise removed), are configured to decrease the cross-sectional area/volume of the blood vessel segment in which the stent is implanted during low-pressure conditions, such as diastole. Reshaping the blood vessel segment to a non-circular cross-sectional shape can serve to force blood through the blood vessel segment by pushing the blood through the vessel as the vessel volume reduces in connection with stent contraction induced by cyclical drops in blood pressure.

Stents of the present disclosure that have a biased non-circular shape (e.g., oval- and/or peanut-shaped) can advantageously be configured to reshape a target blood vessel segment to generate a differential cross-sectional area or volume of the target blood vessel (e.g., aorta) between high- and low-pressure phases of the cardiac cycle to facilitate perfusion. As described above, relatively non-compliant blood vessels generally may not be able to stretch to thereby lengthen the perimeter of the blood vessel in response to increased pressure conditions. Such inability to stretch can prevent compliant expansion of the blood vessel.

As the stents associated with examples of the present disclosure produce complaint blood vessel volume change by manipulating/reshaping the native blood vessel walls, compliance can be increased in the target blood vessel without requiring blood vessel grafting or resection. Therefore, compared to blood flow solutions involving blood vessel grafting/resection, examples of the present disclosure can provide a compliance-enhancing solution that avoids the risks that may be associated with cutting of the vessel and/or devices grafted in/to such vessels, which may present risk of rupture and blood leakage outside of the circulatory system. Hazards associated with extravascular arterial blood leakage, such as within the abdominal and/or chest cavity, can include the risk of serious injury or death.

As described above, desirable diastolic flow in arterial blood vessels is enabled by the decrease in cross-sectional area/volume of the blood vessels when transitioning from higher-pressure conditions (e.g., systole) to lower-pressure conditions (e.g., diastole). Where the relevant blood vessel has become stiff and non-compliant, stretching/expanding and subsequent contraction/shrinking of the blood vessel to cause the desired change in area/volume of the blood vessel may be limited due to the perimeter/wall of the blood vessel being resistant to stretching. Examples of the present disclosure provide implants that cause a change in cross-sectional area/volume of a target blood vessel without requiring stretching in the blood vessel wall. Rather, such cyclical change in blood vessel area/volume can be achieved through manipulation of the shape (e.g., cross-sectional shape) of the target blood vessel, wherein a transition between blood vessel shapes occurring in response to changing pressure conditions can reduce and increase the area/volume of the blood vessel in a cyclical manner to promote more even flow of blood through the blood vessel throughout the cardiac cycle.

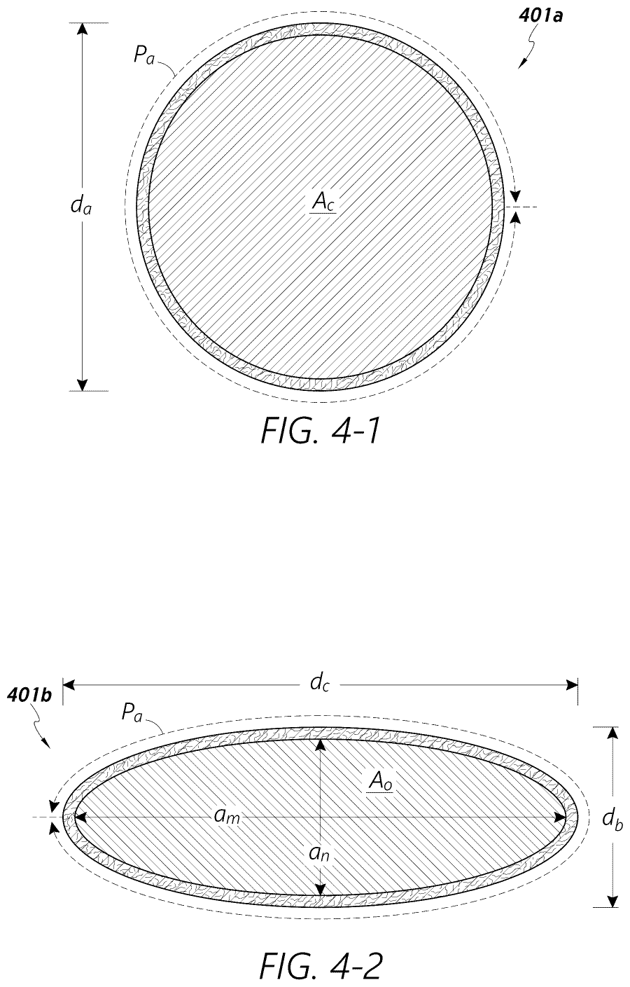

With respect to a blood vessel having a relatively fixed perimeter, wherein the blood vessel wall does not expand sufficiently due to stiffness and/or other factors of non-compliance, generally, the greatest area/volume of the blood vessel may be present/achieved when the blood vessel wall forms a circular cross-sectional shape, which may maximize the cross-sectional area and volume of the blood vessel. FIG. 4-1 shows an example blood vessel 401 (identified as blood vessel 401a in FIG. 4-1) having a generally circular cross-sectional shape, such that the area A_{c} thereof is maximized for the given blood vessel wall perimeter/circumference P_{α}. In the circular configuration, the diameter d_{α} is substantially constant at every angle about the axis of the vessel.

Diverging from a circular cross-sectional shape can produce a cross-sectional area/volume for a blood vessel that is less than the maximum area A_{c} shown in FIG. 4-1. For example, FIG. 4-2 shows the blood vessel 401 (identified as vessel 401b in FIG. 4-2) having a shape that resembles an oval/ellipse, which produces the cross-sectional area Aₒ that is less than the area A_{c} with the same blood vessel wall/perimeter length P_{α}. The oval shape of the vessel 401b may have a major axis aₘ having a dimension d_{c} that is greater than a dimension d_{b} of the minor axis an thereof.

With further reference to FIGS. 4-1 and 4-2, due to the area A_{c} of the oval vessel of FIG. 4-1 being less than the area A_{c} of the circular configuration shown in FIG. 4-1, transitioning from the circular shape 401a to the non-circular shape 401b can provide a reduction in area/volume of the blood vessel 401, and therefore solutions that cause transitions between circular and non-circular blood vessel shapes between cardiac phases can provide compliance characteristics without the need for elasticity in the blood vessel wall tissue. For example, where a mechanism is implemented to cause a blood vessel to transition between circular and non-circular shapes in response to changing pressure conditions, such manipulation of the blood vessel shape can introduce volumetric change in the blood vessel in response to the typical changes in pressure experienced during the cardiac cycle, thereby increasing cardiac efficiency and reducing pulsatile load.

In view of the foregoing, examples of the present disclosure provide stent implant devices and associated processes configured to transition the shape/area of a blood vessel from circular/more-circular to non-circular/less-circular shapes, and vice versa, to enhance compliance with respect to the area of the implant reshaping. Such stent implant devices/processes may effect vessel reshaping through dynamic reshaping of the structural shape of the stent in a way that produces a change in shape of the blood vessel in which it is implanted to produce a change in blood vessel area/volume between the systolic and diastolic phases of the cardiac cycle.

Examples of the present disclosure provide for stent-type implants that are biased to a non-circular cross-sectional area, such that, in a relaxed/non-pressurized state, a first diameter of the stent has a greater dimension along a major axis compared to a second diameter of the stent along a minor axis, wherein such stents are configured to transition to a more-circular shape when pressure within the blood vessel overcomes the non-circular bias of the stent and causes the stent walls to be pushed to the more-circular configuration. The ability of stent implant devices of the present disclosure to reshape the target blood vessel in the manner described above to produce the desired oval cross-section of the blood vessel can be achievable due to stiff/non-compliant blood vessels, which may be unable to stretch to a substantial degree, still retaining the ability to bend to a sufficient degree to allow for such shaping of the blood vessel. That is, the bending stiffness of a non-compliant blood vessel may be relatively lower compared to the stretching stiffness thereof. Therefore, examples of the present disclosure achieve compliance through bending energy with respect to the blood vessel wall, as opposed to stretching energy. When stents of the present disclosure are forced to a circular, or relatively more-circular, axial cross-sectional shape, energy may be stored in the shape memory of the walls of the stent, wherein recoil/contraction of the stent towards its biased, oval/non-circular configuration can return/release energy to the blood circulation. The term “stent” is used herein in accordance with its broad and ordinary meaning and may refer to any device configured to be implanted in a lumen of a blood vessel, the device having a tubular form forming a lumen through which blood can flow.

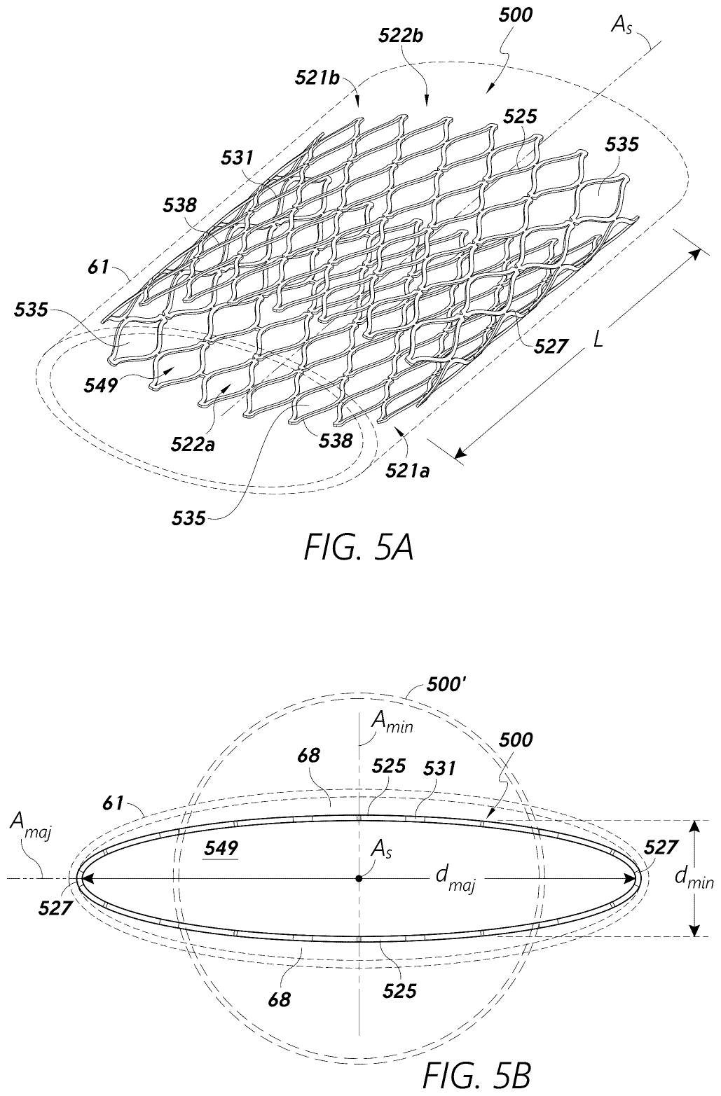

FIGS. 5A and 5B show perspective and axial views, respectively, of a non-circular stent 500 in accordance with one or more examples. The stent may be deployable within a blood vessel lumen. However, it should be understood that example stent devices of the present disclosure may alternatively or additionally be deployable in a position around an outer surface of a target blood vessel. Although not shows for clarity in the figures of the present disclosure, it should be understood that example stents described herein may comprise one or more hooks, barbs, and/or other attachment features/means adapted to facilitate secure attachment of the stent to the tissue of the target blood vessel wall. The description of the stent 500 may be understood to relate to, and/or describe aspects of, any of the stents described herein, wherein such stents can be associated with circularizing/shaping support devices/structures; that is, description of aspects of any example stent and/or associated circularizing/shaping supports of the present disclosure may be understood to be implementable in any other example stent or stent-circularizing/reshaping structure of the present disclosure.

The stent 500 may be formed of a tubular frame 531, which may form a wall around an axial channel 549, thereby defining the channel 549. The stent 500 may be an elongate/elongated stent, in that a length L of the stent is greater than a minor diameter dₘᵢₙ and/or maximum diameter dₘₐⱼ of the stent. As described herein, the frame wall 531 of the stent 500 can be considered a single, circumferentially-wrapped wall, or may be considered to comprise multiple walls, or wall segments. For example, with respect to oval stents and other non-circular stents, as illustrated in FIGS. 5A and 5B, such stents may be considered to comprise sidewall segments 525 that run along relatively long sides of the stent that are aligned generally with the orientation of the major axis/dimension Aₘₐⱼ of the stent, as well as end wall segments 527, which may connect the side walls 525 on major-axis ends of the stent 500. The end walls 527 may be outwardly-curved/concave with respect to an axis Aₛ of the stent 500. The sidewalls 525 may bow/deflect outward, either in a resting, unpressurized state, or in conditions of hoop/wall stress on the frame 531. For example, the sidewalls 525 may bow outward such that the sidewalls 525 are concave from the perspective of the axis Aₛ of the stent 500 and convex from the perspective of the exterior of the stent 500.

Certain stent shapes are described herein, including non-circular-, oval-, peanut-, and other-shaped stents. It should be understood that such description of stent shapes refers to a shape of an axial cross-section of a stent, as depicted in the view of FIG. 5B. Although oval- and peanut-shaped stents are described, it should be understood that the principles of the present disclosure may relate to stents having any non-circular shape in at least some configurations thereof (e.g., in a relaxed/biased configuration), and circularizing/reshaping supports of the present disclosure may be configured to circularize stents having non-circular shapes that are other than the illustrated oval- and peanut-shaped stents. Descriptions of stents in a relaxed or biased configuration should be understood to relate to a configuration that a stent naturally assumes in the absence of tension on the stent wall(s) from external forces (e.g., ambient fluid pressure, physical contact forces, etc.). For example, the biased/relaxed shape of the stent may be due to shape memory of the stent and/or frame thereof.

The stent 500 may be considered an oval stent with respect to the shape of the axial cross-section thereof, as shown in FIG. 5B. The term “oval” is used herein according to its broad and ordinary meaning and may be used substantially interchangeably with the term “ellipse” and/or “oblong,” which terms are likewise used according to their broad and ordinary meanings. The term “oval” may be used to refer to any non-circular closed curve having major and minor axes, the major axis being greater than the minor axis. With respect to “oval”-shaped stents disclosed herein, such stents may have relatively flatter minor-axis sidewalls (compared to curved major-axis end walls), wherein the sidewalls may bow radially outward, and/or may be deflected/curved radially inward so as to produce external concavity and internal convexity in such sidewalls (e.g., forming a peanut-shaped stent). Major-axis walls of an oval stent as described herein may be considered wall portions of a stent that are intersected by a major axis of the stent that runs through an axial center of the stent. Minor-axis walls of such oval stents may be considered wall portions that are intersected by a minor axis of the stent that runs through the axial center of the stent. Example stents of the present disclosure may be considered to have an oval shape whether or not the shape thereof is definable by an algebraic curve. Example stents of the present disclosure may be considered oval stents when the wall(s) of the stent in an axial-cross-sectional perspective form(s) a closed or open curve in a plane Pₛ that is non-circular; one or more segments/areas thereof may resemble the outline of a portion of an egg. Oval stents of the present disclosure may include either one or two axes of symmetry of an ellipse, such as the illustrated major Aₘₐⱼ and minor Aₘᵢₙ axes. The axial cross-section of some examples of oval stents of the present disclosure may resemble the union of two semicircles on opposite sides of a rectangle, providing a shape evoking the likeness of a speed skating rink or an athletics track. In some contexts, the oval stent 500 may be referred to as a “stadium”-shaped stent, or an elongated oval.

The stent frame 531 comprises stent wall(s) defining an elongated tubular structure having a first axial end 521a with a first opening 522a. The tubular structure may further comprise a second axial end 521b with a second opening 522b, wherein the lumen/channel 549 extends between the first opening 522a and the second opening 522b, traversing the length L of the stent 500. The frame 531 and/or wall(s) thereof may comprise an open-cell structure adapted to be expanded to secure the stent 500 to a blood vessel internal (or external) wall, such as through endothelialization of the frame 531 to the vessel tissue over time as the frame 531 is held in a generally-circular form by one or more circularizing/reshaping support structures, such as a circular stent and/or major-axis tether/tie.

The stent 500 may be elastically deformable between a first, non-circular configuration and a second, more-circular configuration 500′ (see dashed-line representation in FIG. 5B), with the stent 500 biased toward the non-circular configuration. In some examples, the stent frame 531 may comprise a shape-memory material, such as Nitinol. Although shown as an oval-shaped stent, the stent 500 may be any non-circular shape in a relaxed state thereof, such as a triangle, peanut, figure-8, star, clover/lobed, and/or kidney shape.

The stent 500 may be configured to be percutaneously delivered to a blood vessel 61 in a compressed delivery configuration. Once within the blood vessel lumen at the target deployment site, the stent 500 may be configured to be radially expanded into direct surface contact with the blood vessel wall (e.g., the inner wall of an aorta segment). In some examples, the stent 500 may be configured to be expanded such that the perimeter of the stent 500 approximates and/or exceeds a perimeter of the blood vessel portion where the stent 500 is implanted, at least immediately prior to deployment/expansion of the stent. Placement of the stent 500 in the blood vessel may cause at least slight stretching in the blood vessel wall, such as due to pressure at the major-axis ends 527 against the blood vessel wall. In some cases, a stent configured to expand to a greater perimeter than the native blood vessel may provide improved traction and/or resistance to migration within the blood vessel. Implanting a stent that has a perimeter approximate to and/or slightly greater than the blood vessel perimeter may increase positive engagement with the blood vessel wall and/or maximize a compliance effect. The stent wall and/or a portion thereof may be configured to be endothelialized to the blood vessel wall.

In the oval configuration shown in FIGS. 5A and 5B, the stent 500 may have a cross-sectional area having a major/long-axis diameter dₘₐⱼ that is substantially larger than the minor/short-axis diameter dₘᵢₙ. For example, the major-axis diameter/dimension dₘₐⱼ may advantageously be at least twice as long as the minor-axis diameter/dimension dₘᵢₙ, or even 3, 4, 5, 6, or 7 times greater. The stent 500 may be configured to increase compliance of a blood vessel though constant or near-constant pressure at one or more points along a perimeter/circumference of the blood vessel that causes a change in the perimeter geometry of the vessel. For example, the blood vessel may be changed and/or moved from a non-circular/less-circular shape to a circular/more-circular shape.

The stent frame wall(s) 531 may be at least partially composed of struts 538 and/or stent openings/cells 535 between the struts 538. The dimensions and/or shape of the stent 500 may vary based on the particular application and/or target implantation anatomy. For example, the stent length L may be selected to extend over all or a portion of an identified non-compliant length of a target blood vessel. The stent major axis dₘₐⱼ and minor axis dₘᵢₙ, when averaged, may be approximately equal to the diameter of the native blood vessel. For example, for a stent configured for deployment in an aorta, the length L may be between 1-30 cm, and in the biased oval/diastolic configuration the major axis dₘₐⱼ may be between 1-4 cm (or larger/smaller depending on the particular anatomy), and the minor axis dₘᵢₙ can be between 20-50 percent of the major axis dₘₐⱼ. However, other sizes and/or shapes are also within the scope of this disclosure.

The configuration of the stent 500 in the oval shape can cause blood vessel wall 61 to assume a more oval shape to match the shape of the stent 500. However, depending on the relative size of the stent 500 to the vessel 61, the blood vessel 61 may not necessarily conform exactly to the circumference and/or shape of the stent 500, and gap(s) 68 may be present and/or form between the frame 531 and the blood vessel wall 61 as the luminal pressure increases and pushes the vessel side walls away from the frame sidewall 525. Due to the presence of the gaps 68, which may form cyclically as the pressure drops (e.g., during diastole) and the stent transitions to the oval shape, such that the walls 525 pull farther away from the blood vessel wall, desirable sealing between the stent and the blood vessel may be impeded. The presence of the gaps 68 can reduce the ability of the stent 500 to reshape the blood vessel, thereby negatively impacting the efficacy of the stent 500 with respect to compliance-enhancement.

When implanted in a blood vessel, the patient physiology may respond to the stent 500 as a foreign object. For example, macrophages can accumulate around the stent, and nearby smooth muscle cells can proliferate to cover the stent. Over time, a new endothelial layer can form over the stent, which can inhibit clot formation. In addition to preventing thrombus formation, endothelialization can enhance the ability of the stent to reshape the target blood vessel by strengthening the physical coupling between the stent and the blood vessel, thereby reducing the presence of gaps forming between the stent and blood vessel wall when the stent walls pull away from the blood vessel wall as the stent reshapes to an oval/non-circular shape. Tissue overgrowth can be promoted through contact between the stent frame and the blood vessel walls. Examples of the present disclosure provide devices that facilitate contact between non-circular stent frames and blood vessel walls, thereby increasing the efficacy of the stents with respect to reshaping/compliance-enhancement.

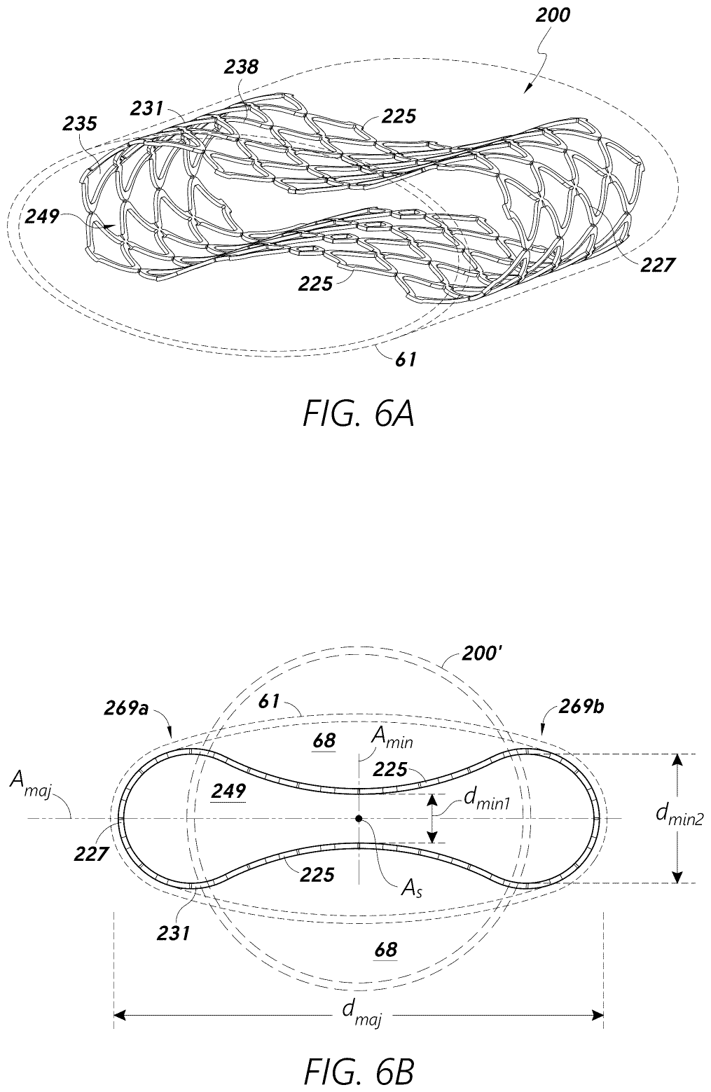

FIGS. 6A and 6B show perspective and axial views, respectively, of a non-circular stent 200 having inwardly-deflected sidewalls 225 in accordance with one or more examples. The stent 200 may represent an example implementation of any of the non-circular/oval stents disclosed herein, or portion(s) thereof. The stent 200 forms/defines an elongate/elongated tubular member, as shown in FIG. 6A, which forms a blood flow lumen 249.

The shape of the stent 200 deviates from the stent 500 shown in FIGS. 5A and 5B only in that the major-axis (e.g., relatively flat and/or long) sidewalls 225 deflect to a greater degree towards the center/axis Aₛ of the stent 200 in a relaxed state. The resulting shape may resemble that of an hourglass and/or peanut shape with respect to the axial cross-section shown in FIG. 6B. The stent 200 may have a major axis diameter/dimension dₘₐⱼ that is greater than a minor axis diameter/dimension dₘᵢₙ₁, dₘᵢₙ₂ of the oval cross-section.

In some examples, the stent 200 may be biased to a shape having a minor axis dimension dₘᵢₙ that is non-constant along the major axis Aₘₐⱼ dimension, which forms an externally-concave/internally-convex surface/form with respect to the minor axis sidewalls 225. For example, the minor axis sidewalls 225 may have a diameter dimension dₘᵢₙ₁ at a center thereof (with respect to the major axis dimension Aₘₐⱼ) that is less than the diameter/dimension dₘᵢₙ₂ at/towards the end portions of the minor-axis sidewalls 225. Non-circular stents of the present disclosure that have externally-concave/internally-convex minor-axis sidewalls as shown in FIGS. 6A and 6B are referred to herein as peanut-shaped stents; such peanut shape can be considered a variation of an oval, or oval-shaped, stent as described herein. The inwardly-deflected walls 225 can allow for a transition reshaping between peanut-shaped, to outwardly-bowed oval shape, and ultimately to circular/more-circular shape, shows as the dashed circular stent shape 200′.

The stent 200 that may be biased (such as being memory-set via a memory material such as nitinol) toward the illustrated peanut shape. With the sidewalls 225 deflected inwardly, as shown, the stent frame 200 may form bulging/bulbous portions 269a, 269b on either side of the minor axis Aₘᵢₙ, wherein such portions have a diameter/dimension dₘᵢₙ₂ that is greater than the shortened diameter dₘᵢₙ₁. Compared to the oval stent 500 of FIGS. 5A and 5B, the peanut-shaped stent 200 may have a tendency to form even greater gaps 68 between the minor-axis sidewalls 225 and the blood vessel walls 61 due to the inward deflection of the walls 225 away from the blood vessel wall. Therefore, peanut-shaped stents may suffer to a relatively greater degree from impediments to blood vessel reshaping and compliance enhancing without desirable tissue growth coupling the sidewalls 225 to the blood vessel 61. Circularizing support stents, ties, and other structures of the present disclosure can improve blood vessel coupling of peanut-shaped stents like the stent 200 of FIGS. 6A and 6B.

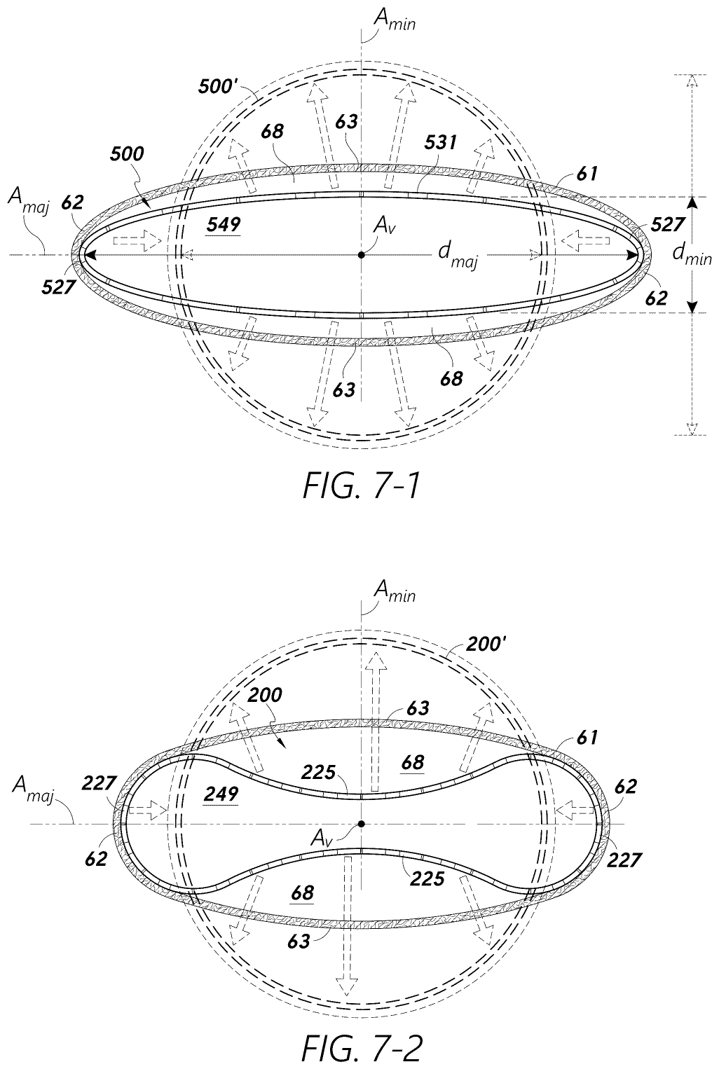

FIG. 7-1 shows an axial view of the oval stent 500 shown in FIGS. 5A and 5B deployed within a blood vessel 61 in accordance with one or more examples. The stent 500 may be biased toward the illustrated oval and/or other non-circular relaxed/diastolic configuration (shown in solid-line), and may, when subjected to mechanical forces associated with high luminal pressure, be configured to responsively transform to a more-circular systolic configuration (shown in dashed-line) such that the minor axis dₘᵢₙ approaches, and may equal, the major axis dₘₐⱼ. As with any of the examples disclosed herein, the stent 500 can be configured to deflect from the oval shape to a more-circular shape in the presence of threshold blood pressure levels greater than 80 mmHg, such as blood pressure levels greater than 90 mmHg (e.g., between 90-120 mmHg).

For examples in which the stent frame 531 is not covered by a fluid-tight covering, the cells of the frame 531 can provide openings in the frame 531 that allow blood in the blood vessel 61 to transfer pressure through the frame 531, to thereby load the inner diameter/surface of the blood vessel with a force resulting from increases in the luminal blood pressure as the heart beats. For example, luminal pressure forces against the blood vessel wall increase the hoop stress on the blood vessel, which may force the blood vessel, and with it the stent 500, to assume a more-circular shape. The resulting hoop stress from luminal pressure increase can exert radially-outward force along the blood vessel's inner circumference, such stresses/forces being tensile in nature, which can tend to cause the blood vessel to increase in diameter. The blood pressure force on the blood vessel wall and resulting inward deflection of the blood vessel wall (due to outward deflection of the vessel wall portions in the area of the minor axis Aₘᵢₙ) at the major-axis-ends 527 of the stent 500 may cause inward deflection of the ends 527 of the stent 500 to form a desired geometric change to a more-circular shape 500′ (shown in dashed line) of the stent and the blood vessel 61. The transition of the stent from oval to the more-circular stent shape 500′ (shown in dashed-line in FIG. 7-1) causes energy to be stored in the stent frame 531 (e.g., in the elasticity and/or shape memory thereof), such that energy is returned to the blood vessel walls, and therefore to the blood circulation within the blood vessel segment, when the frame transitions back to the oval shape 500 as pressure decreases.

The natural cross-sectional shape of the aorta (and other blood vessels) may generally be circular. With the oval stent 500 pushing outward to the oval configuration, the wall portions 63 may be pulled/drawn at least partially towards an axial center Aᵥ of the blood vessel 61 and/or towards each other in a manner as to cause the blood vessel 61 to form a non-circular shape, such as the oval shape shown in FIG. 7-1. However, depending on the relative size of the stent 500 to the vessel 61, the blood vessel 61 may not necessarily conform exactly to the circumference and/or shape of the oval stent 500, and gap(s) 68 may be present and/or form between the frame 531 and the blood vessel wall 63 as the luminal pressure increases and pushes the vessel side walls 63 away from the frame sidewall 525.

The stent frame 531 may be inclined to experience tissue in-growth in one or more areas thereof. For example, the end walls 527 may be relatively stationary relative to the blood vessel walls 62 throughout the cardiac cycle due to the relatively constant contact between the stent side walls 527 and the blood vessel sidewalls 62 between oval and circular shapes of the stent 500. Conversely, in the absence of sufficient tissue growth coupling the stent sidewalls 525 to the vessel sidewalls 63, the sidewalls 525 of the frame 531 and the sidewalls 63 of the blood vessel may tend to come into contact with one another and separate in a cyclical manner as the blood pressure forces the blood vessel sidewalls 63 outward and away from the stent sidewalls 525 during portion(s) of the high-pressure phase/stage of the cardiac cycle. Therefore, tissue ingrowth may occur primarily between the blood vessel end walls 62 and the stent end walls 527 in situations in which tissue ingrowth occurs.

For examples in which the stent frame 531 is covered internally and/or externally by a fluid-tight covering (not shown in FIG. 7-1 for visual clarity), the openings of the cells 535 of the frame 531 may be closed to pass-through fluid and prevent blood in the blood vessel 61 and within the channel 549 of the stent 500 from transferring pressure through the frame 531. Therefore, intraluminal pressure within the flow channel 549 of the stent 500 loads against the frame 531 (rather than directly against the blood vessel walls 63) to provoke reshaping thereof. The blood-pressure-induced force against the covering and/or stent frame 531 can increase the hoop stress on the frame 531 and/or covering, which may force the frame 531, and with it the blood vessel 61, to assume a more-circular shape. Unlike with bare-frame stents that do not include fluid-tight coverings, where a covering is present, the endoluminal pressure forces in the channel 549 of the covered stent 500 are distributed over/against the covering and/or frame 531 to directly reshape the stent 500, rather than indirectly reshaping the stent 500 through force on a finite number of end contact/pressure points of the frame 531. As the pressure in the channel 549 increases (e.g., in connection with the systolic phase of the cardiac cycle), the plastically-deformable nature of the stent frame 531 allows for the sidewalls 525 of the frame 531 to be pushed outward along with the shortening of the stent 500 in the major axis dimension Aₘₐⱼ. When the sidewalls 525 are deflected outward, the channel 549 assumes a more circular cross-sectional shape.

With respect to covered stent examples, the biasing/shape-memory of the stent frame 531 may cause the sidewalls 525 to naturally pull towards the center of the frame 531, thereby potentially pulling away from the vessel sidewalls 63 to form gap(s) 68 between the stent walls 525 and the vessel walls 63 if insufficient tissue coupling between the stent 500 and the vessel 61 has occurred. Therefore, as with bare-frame stent examples, it may be desirable to promote tissue ingrowth between the covered non-circular stents and a blood vessel by forcing a more-circular shape thereof to a time sufficient to permit the desired tissue ingrowth to occur, which promotes physical coupling between the stent and the blood vessel.

FIG. 7-2 shows an axial view of the peanut-shaped stent 200 shown in FIGS. 6A and 6B deployed within a blood vessel 61 in accordance with one or more examples. Once deployed within a blood vessel, during diastolic pressure, the stent sidewalls 225 may remain at least partially inwardly deflected or, due to blood pressure levels present (even during low-pressure conditions, e.g., diastole), the stent 200 and/or blood vessel 61 may deform into a substantially oval, non-peanut shape. When subjected to the higher pressures of systole, the stent 200 and/or blood vessel 61 may deform to a more circular shape.

The peanut-shaped stent 200, when implanted in a blood vessel 61, may cause similar reshaping of the blood vessel 61 as the oval stent 500 described above. However, due to the inward deflection of the minor-axis sidewalls 225, the gaps 68 between the sidewalls 225 and the blood vessel sidewalls 63 may be greater with respect to at least some periods or stages of the cardiac cycle and/or when the stent 200 is initially deployed within the blood vessel 61. That is, the tendency of the stent sidewalls 225 to pull away from the blood vessel sidewalls 63 may be relatively high, and therefore, without tissue overgrowth coupling the vessel walls 63 to the stent walls 225, the reshaping ability/function of the stent 200 may be at least partially impaired/impeded. Furthermore, where the gaps 68 are present between the stent walls 225 and the vessel walls 63, blood may collect and stagnate in such spaces, presenting embolism risk and/or impeding outward deflection of the stent walls 225 to circularize during the systolic phase of the cardiac cycle. Therefore, facilitating tissue overgrowth of the stent walls 225 may be of particular significance with respect to peanut-shaped stents.

For bare-frame peanut-shaped stent configurations, the gaps 68 between the stent walls 225 and the vessel walls 63 may form as the luminal pressure increases and pushes the vessel side walls 63 away from the frame sidewalls 225, which in-turn forces the end walls of the vessel 62 and the end walls 227 inward to cause the stent sidewalls 225 to outwardly deflect to the more-circular shape 200′ shown in dashed-line representation in FIG. 7-2. For covered peanut-shaped stent configurations, increases in luminal pressure may directly push against the sidewalls 225 (and/or covering) to cause outward deflection thereof. For covered and non-covered implementations, as pressure decreases, the shape memory of the frame 231 causes the sidewalls 225 to pull back away from the vessel walls 63 to form the gaps 68.

In order to promote tissue ingrowth between sidewalls of non-circular (e.g., oval, peanut-shaped) stents of the present disclosure and blood vessel walls, certain support structures/devices can be implemented to force the shape of a non-circular-biased stent to a cross-sectional shape that is more circular than the biased/relaxed shape thereof, such that the stent walls are more evenly/consistently in contact with and/or proximate to the walls of the target blood vessel for a sufficient period of time to allow for the desired tissue overgrowth to occur. Such stent-shaping/circularizing supports can be configured to reshape the stent for only a transitory/temporary period of time sufficient to allow for the desired tissue overgrowth to occur, after which point the support(s) may dissolve or otherwise be removed from connection/contact with the non-circular stent to allow the stent to return towards a biased non-circular shape thereof.

Tissue overgrowth of a non-circular stent that is forced to a more-circular shape by support structure(s) of the present disclosure can involve endothelialization of the blood vessel wall over the stent frame and/or covering associated therewith. Generally, the deployment of the stent in the target blood vessel can cause injury to the vascular wall and endothelium, which comprises a layer of endothelial cells lining the vascular wall that promotes homeostasis. The injury to the blood vessel wall can result in inflammation, repair, and the development of neointimal hyperplasia. The ability of the endothelium to repair itself can depend on the migration of surrounding mature endothelial cells, and the attraction and adhesion of circulating endothelial progenitor cells (EPCs) to the area where the stent is deployed, forming overgrowth of endothelial-like cells.

In some examples, stent frames and/or coverings can be implemented with drug-eluting features/coatings that at least partially interrupt the natural tissue overgrowth process. Alternatively, stents can be implemented with treatments that accelerate the reendothelialization of the damaged arterial segment following stent deployment. Such tissue-growth promotion can reduce risks of neointimal hyperplasia and stent thrombosis. In order to promote the desired coupling between stent walls and blood vessel walls to facilitate blood vessel reshaping as described in detail herein, stent frames and/or coverings of stents of the present disclosure can be treated with agents that augment the generation and/or accumulation of endothelial cells or EPCs in the area of the deployed stent. In some implementations, the stent frame and/or covering can be seeded with endothelial cells or EPCs. For example, a stent can be coated with anti-CD34 antibodies to attract endothelial/EPC cells to the stent. The stent-reshaping/circularizing structures disclosed herein can be designed to hold the associated stent in a relatively circular shape for a period of time that correlates with the expected tissue overgrowth period based on the tissue-growth-promoting/inhibiting features of the stent implant.

Stent-circularizing support structures of the present disclosure can advantageously be biodegradable, such that after a period of time, such structures cease forcing a more-circular shape of the stent, thereby allowing the stent to assume/revert-to a biased non-circular shape to thereby reshape the target blood vessel. Such reshaping of the target blood vessel advantageously occurs after the support structure has held the stent in a relatively circular shape to promote and allow for tissue ingrowth and coupling between the stent and the blood vessel. Stent-circularizing supports of the present disclosure can take the form of stents, ties/tethers, or other forms.

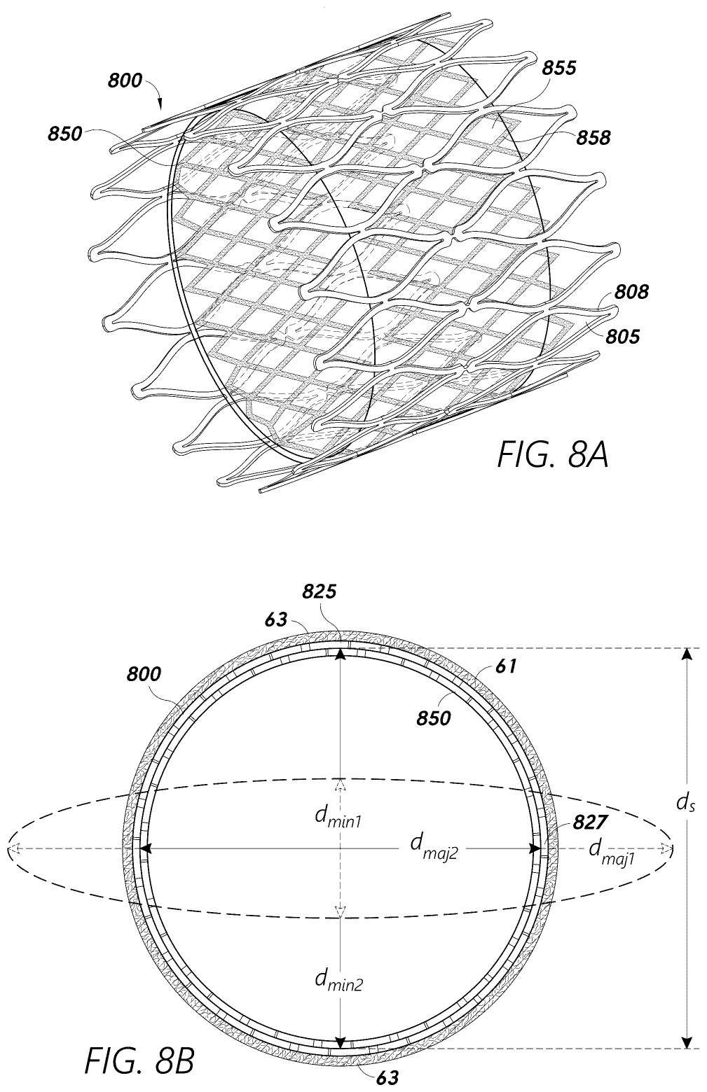

FIG. 8A shows a perspective view of a biodegradable circularizing support stent 850 in accordance with one or more examples of the present disclosure. FIG. 8B shows an axial view of the circularizing support stent 850 deployed within a non-circular stent 800 (e.g., oval- or peanut-shaped stent) within a blood vessel 61 in accordance with one or more examples. The support stent 850 is shown as a stent structure formed of an arrangement of struts 858 arranged to form open cells 855, thereby providing a relatively minimalistic structure. However, it should be understood that circularizing support stents of the present disclosure may comprise solid/planar walls/sheets of material, rather than the open-cell implementation shown.

The support stent 850 advantageously has a substantially circular biased axial shape, as shown. The support stent 850 may be configured to be radially compressed to a low-profile delivery configuration, wherein upon deployment from a delivery system (e.g., catheter/sheath) utilized to deliver the implant 850, the support stent 850 may self-expand, or may be balloon-expanded or otherwise mechanically expanded using certain instrumentation to the circular shape shown in FIGS. 8A and 8B. Such expansion of the sacrificial/removable support stent 850 may occur when the support stent 850 is within an internal diameter/channel of the outer, non-circular stent 800. In some implementations, the sacrificial support stent 850 and the outer non-circular stent 800 can be expanded together, such as simultaneously and/or using the same instrumentations. For example, radial expansion of the support stent 850 within the outer stent 800 can force the radial expansion of the outer stent 800 as well.

As referenced above, the support stent 850 may be deployed within the internal channel of the non-circular stent 800. The support stent 850 may be expanded to the circular configuration shown in FIG. 8B, wherein the circular stent 850 has a substantially constant diameter dₛ. The diameter dₛ of the support stent 850 may be greater than a biased minor-axis dₘᵢₙ diameter of the non-circular stent 800, wherein expansion of the support stent 850 within the stent 800 causes the minor-axis diameter of the stent 800 to expand to an expanded minor-axis dimension dₘᵢₙ₂, as shown, which may be approximately equal to the outer diameter dₛ of the support stent 850. Furthermore, the expansion of the support stent 850 within the non-circular stent 800, by deflecting minor-axis sidewalls 825 thereof outwardly, may cause the inward deflection of the end walls 827 along the major axis of the stent 800, thereby reducing the major axis dimension dₘₐⱼ of the stent 800 to approximate the outer diameter dₛ of the support stent 850.

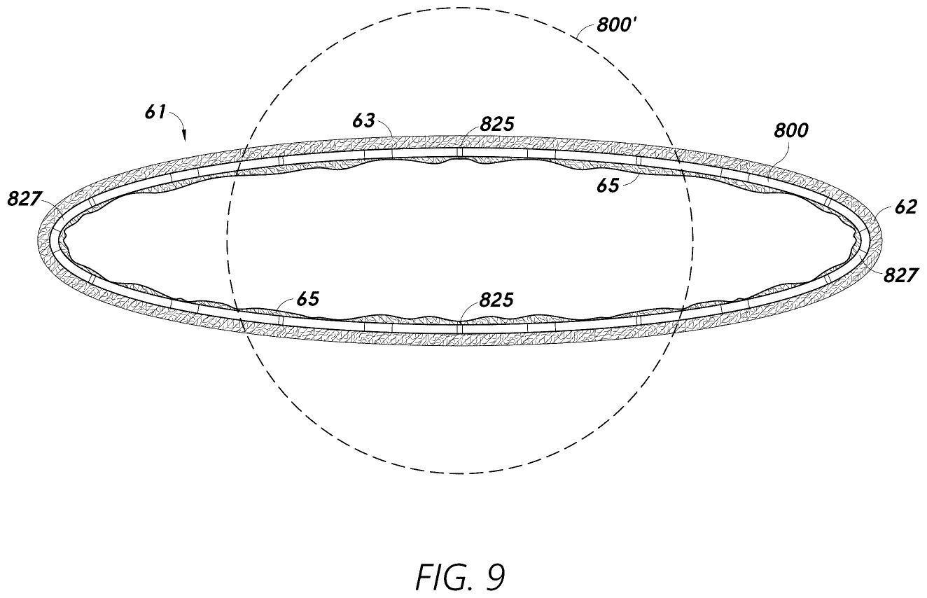

The support stent 850 may be configured to hold the circular shape of the stent 800 as shown in FIG. 8B for a period of time sufficient to allow tissue overgrowth 65 (see FIG. 9) to couple the stent 800 to the blood vessel 61. For example, by pushing the sidewalls 825 outward and against the walls 63 of the blood vessel 61, tissue overgrowth 65 may be permitted/facilitated between the walls 63 and the stent sidewalls 825, to thereby physically/mechanically couple the sidewalls 825 to the blood vessel walls 63. At least for a period prior to dissolution or removal of the support stent 850, tissue growth may cover portions of the support stent 850, such as within open cells or other features thereof. The tissue growth 65 may project/accumulate through the open cells 805 of the stent 800, and may span across struts 808 of the stent frame on an internal diameter of the frame. In some implementations, the growth 65 may accumulate on a covering of the stent 800. After a sufficient period of time (e.g., eight hours or more, one or more days), the sacrificial support stent 850 may be removed or may dissolve of its own accord. For example, the support stent 850 may comprise biodegradable material configured to at least partially dissolves and/or otherwise break-down over time, thereby defeating the circularizing structural support of the support frame 850.

FIG. 9 shows the non-circular stent 800 within the blood vessel 61 after the support stent 850 has been dissolved and/or removed and tissue overgrowth 65 has occurred, binding the sidewalls 825 of the stent 800 to the walls 63 of the blood vessel 61. As shown, after sufficient tissue overgrowth 65 has occurred, when the non-circular stent 800 reverts to its biased non-circular shape (e.g., oval-, peanut-shaped), the sidewalls 63 of the blood vessel may be inclined to deflect radially inwardly along with the sidewalls 825 of the stent 800, such that little or no gap is present between the sidewalls 825 and the blood vessel wall 63. In such configuration, cyclical reshaping of the stent 800 and blood vessel 61 between oval and circular shapes may occur in tandem. For example, in such configuration, major-axis elongation of the stent 800, as shown in FIG. 9, may cause radially-inward deflection of the sidewalls 825 thereof, which in-turn may pull the walls 63 of the blood vessel 61 radially inward to reshape the blood vessel 61 to a non-circular (e.g., oval) shape. Furthermore, as pressure increases in the blood vessel 61, such pressure forces may push outwardly on the blood vessel walls 63, the stent 800, and/or the tissue growth 65, wherein such forces may deflect the vessel walls 63, thereby pushing and/or pulling the stent walls 825 radially outwardly due to the tissue-coupling therewith, such that the reshaping of the blood vessel 61 to a more-circular shape in high-pressure conditions more efficiently reshapes the stent 800′ to a more-circular shape (e.g., circular). Therefore, the tissue overgrowth facilitated by the implementation of the support stent 850 can improve the efficiency and/or efficacy of the reshaping non-circular stent 800 for the purpose of promoting blood flow and/or enhancing compliance and/or perfusion characteristics associated with the target blood vessel 61.

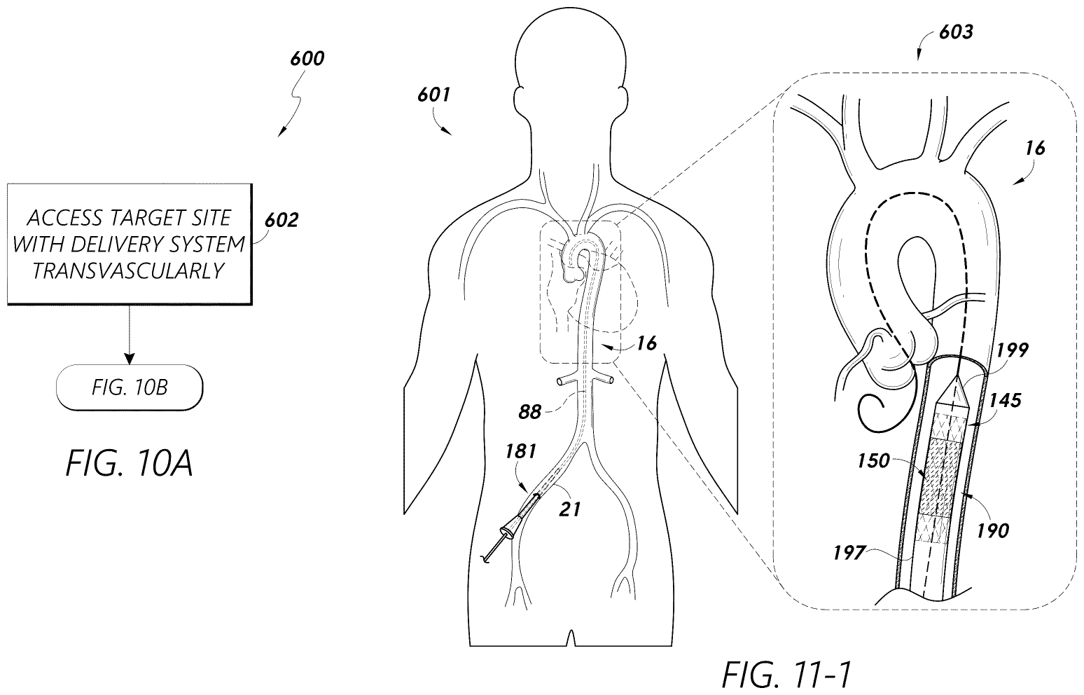

FIGS. 10A-10C illustrate a flow diagram for a process 500 for coupling sidewalls of a non-circular stent 145 to blood vessel walls using a circularizing support stent 150 in accordance with one or more examples. FIGS. 11-1, 11-2A, 11-2B. 11-3, 11-4A, and 11-4B provide images of the stent 145, support structure 150, certain delivery system components, and certain anatomy corresponding to operations of the process 600 of FIGS. 10A-10C according to one or more examples.

At block 602, the process 600 involves advancing a delivery system 190 to a target position in a blood vessel 16, such as the aorta. For example, the delivery system 190 may be advanced through a percutaneous introducer or other minimally-invasive access 181 into the vasculature of the patient, and further within the vasculature to a target position within the aorta 16 of the patient. The delivery system 190 may include one or more catheters/sheaths 197 and/or a nosecone 199 or other feature configured to facilitate the forward advancement of the delivery system 190 through tortuous anatomy of the vasculature. The percutaneous entry 181 may be at the femoral artery or other arterial blood vessel.

The non-circular stent 145 may be transported to the target implantation site in a delivery configuration in which the frame of the stent is radially compressed, as shown in image 603. In some implementations, the circularizing support stent 150 is configured to be transported in a delivery configuration within an inner diameter/lumen of the stent 145, which may allow for simultaneous deployment of the stent 145 and support structure 150 and/or may otherwise simplify delivery of such devices. Alternatively, the stent 145 and support structure 150 may be delivered to the target anatomy in separate delivery systems, and/or in axially offset positions within a common sheath or other delivery system component (e.g., catheter/sheath 197).

At block 604, the process 600 involves deploying the non-circular stent 145 (e.g., oval or peanut-shaped stent) from the delivery system 190, wherein the stent 145 is configured to assume a non-circular shape in a relaxed, non-pressurized state thereof. As referenced above, the stent 145 may be transported to the implantation site in a radially crimped/compressed configuration. The stent 145 may expand in accordance with self-expansion when deployed from the delivery system 190, or may be expanded using a balloon catheter or similar device.

At block 606, the process 600 involves deploying the sacrificial support stent 150 with in the non-circular stent 145 to force the non-circular stent 145 into a circular shape, such as by forcing outward deflection of the minor axis sidewalls 146 of the stent 145, thereby causing major-axis ends 147 to deflect radially inwardly. For example, the support stent 150 may be transported to the deployment site in a compressed, low-profile configuration within the delivery system 190, or within another delivery system, and may be expanded within the interior lumen/channel of the stent 145. Such expansion of the support stent 150 may be performed substantially simultaneously with the expansion of the outer, non-circular stent 145. For example, both stents 145, 150 may be deployed from the delivery system 190 simultaneously, with the support stent 150 disposed within the stent 145, wherein expansion of both stents may be achieved through self-expansion enabled by shape-memory characteristics of the respective stents, or through balloon expansion from within the axial channel of the support stent 150. That is, a balloon catheter or similar device may be disposed within the channel of the support stent 150, wherein expansion of the balloon device causes the support stent 150 to radially expand, which in turn forces the radial expansion of the outer stent 145.

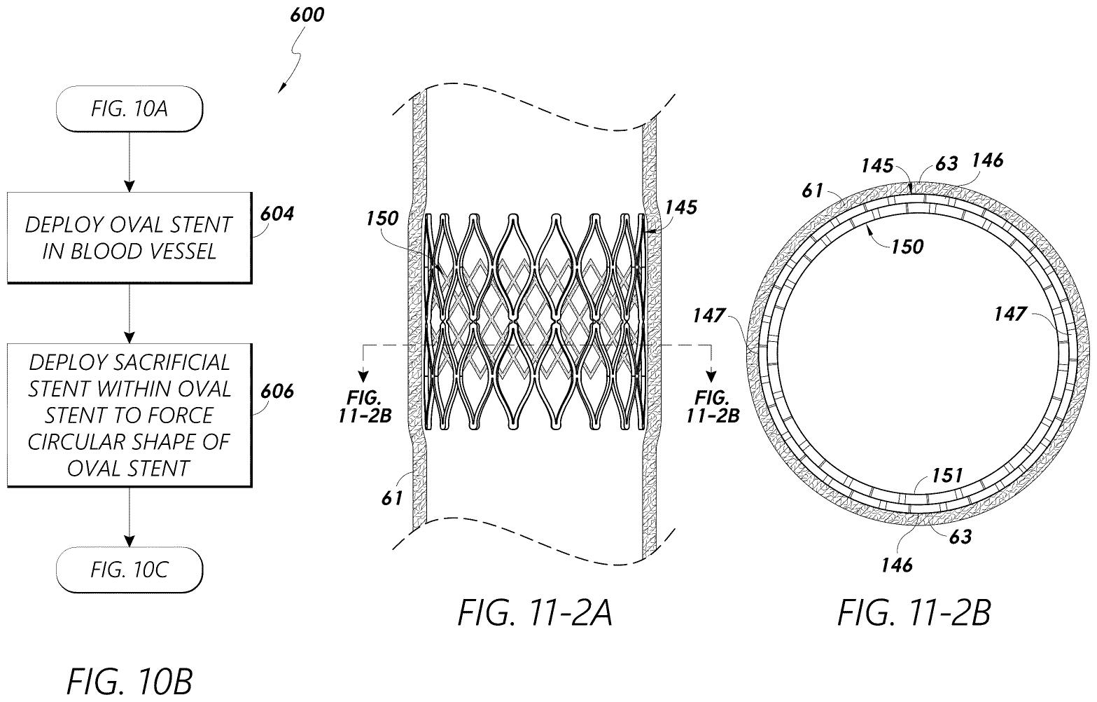

FIG. 11-2A shows the outer stent 145 and the inner support stent 150 deployed in the blood vessel 16, whereas the FIG. 11-2B provides an axial cross-sectional detail of the stents 145, 150, with the support stent 150 forcing the outer non-circular stent into a generally-circular shape, thereby approximating the sidewalls 146 of the stent 145 to adjacent walls 63 of the blood vessel 16 to facilitate tissue overgrowth 65 to couple the stent walls 146 to the blood vessel walls 63 over time.

At block 608, the process 600 involves maintaining the sacrificial support stent 150 in position within the outer stent 145 until the sacrificial stent 150 dissolves, either wholly or at least in part, such dissolution advantageously occurring primarily after formation of the tissue overgrowth 65 that binds the stent 145 to the blood vessel 61. For example, the sacrificial stent 150 may comprise biodegradable material that is configured to dissolve in the presence of the blood medium within the blood vessel 16. In some implementations, the support stent 150 may not fully dissolve, but rather certain portions or components thereof may dissolve or break in a manner as to reduce the structural integrity of the sacrificial stent 150 and/or the ability of the stent 150 to force the circular shape of the non-circular stent 145, thereby allowing the stent 145 to break free from the forced circular state and assume a more natural, non-circular shape thereof (e.g., oval).

At block 610, the process 600 involves reshaping the blood vessel 16 to an oval or other non-circular cross-sectional shape using the stent 145. With the support stent 150 dissolved, the non-circular stent 145 may be permitted to return to its more natural configuration/shape, at least during periods of relatively low pressure within the blood vessel lumen. FIGS. 11-4A and 11-4B shows the stent 145 having assumed a non-circular cross-sectional shape. Due to the tissue overgrowth 65, the sidewalls 63 of the blood vessel 61 may be adhered/coupled to the sidewalls 146 of the stent 145, such that the inward deflection of the sidewalls 146 pulls the blood vessel walls 63 inward in a commensurate manner, such that the stent 145 reshapes the blood vessel to a non-circular shape, as shown.

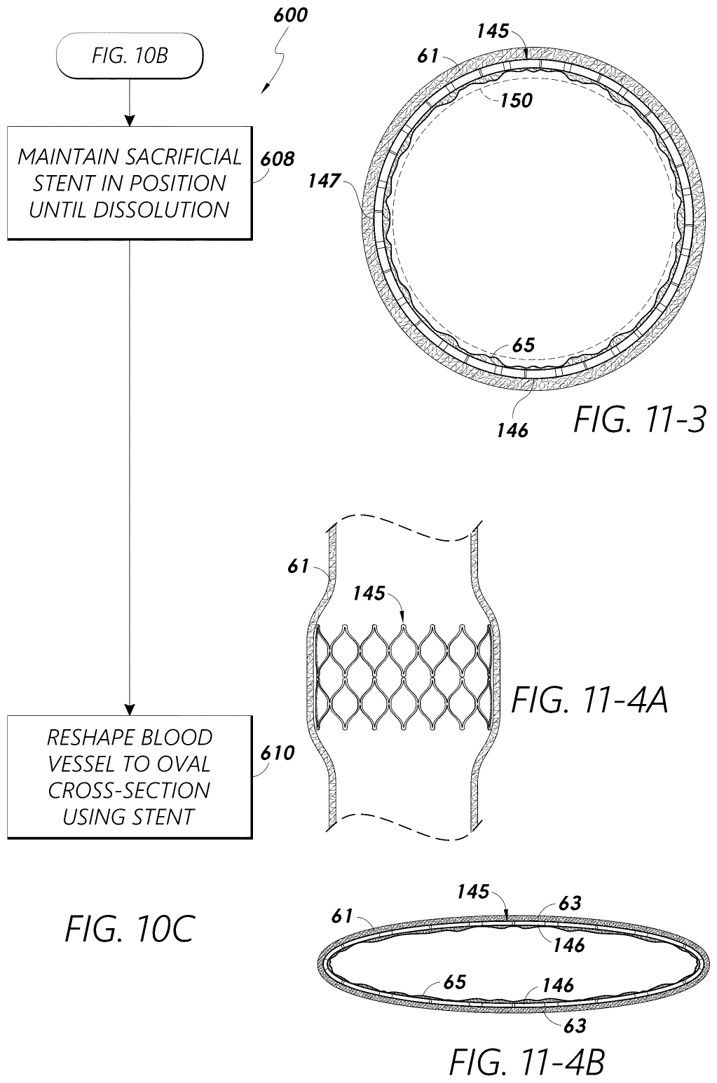

FIG. 12A shows a perspective view of a stent 900 having a non-circular biased shape, wherein the stent 900 is held in a circular shape (or more-circular than the biased shape thereof) by a circularizing tether/tie feature 970, which may or may not be biodegradable. FIG. 12B shows an axial view of the circularizing tie 970 coupled to, integrated with, or otherwise associated with, the stent 900 (e.g., oval- or peanut-shaped stent) within a blood vessel 61 in accordance with one or more examples. The stent 900 is shown as a stent structure formed of an arrangement of struts 948 arranged to form open cells 955. However, it should be understood that circularizing ties/tethers of the present disclosure may be implemented in stents/tubes formed of solid/planar walls/sheets of material, rather than the open-cell implementation shown.

The term “tie” is used herein according to its broad and ordinary meaning and may refer to any elongate cord, suture, strip, tether, strand, line, rope, wire, filament, string, ribbon, strap, or portion thereof, or other type/form of material used in medical procedures (e.g., ePTFE suture, for example, GORE-TEX® sutures, W.L. Gore, Newark, Delaware). Furthermore, in some contexts herein, the any of the tie-related terms listed above may be used substantially interchangeably. In addition, use of the singular form of any of the tie-related terms listed above may be used to refer to a plurality of ties (or similar), a single tie (or similar), or a portion thereof. Any ‘tie’ or similar component disclosed herein can be a tie structure, which may be non-rigid/flexible, rigid, semi-rigid, and/or adjustable. In some implementations, ties/tic-structures disclosed herein can be configured to be adjusted, removed, or unlatched by a secondary catheter/system or other instrumentation after tissue ingrowth has taken place.

The tie 970 may or may not have lengthwise elasticity. It may be desirable to implement the tie 970 as not being elastic/stretchable so as to provide relatively improved control over the forced shape of the stent 900 when the tie is coupled to the stent 900. The tie 970 may be tensioned to hold the stent 900 in a generally-circular axial shape, as shown in FIG. 12B. For example, the tie 970 may be configured to a length d₁ that corresponds to a circular diameter dₘₐⱼ₂ of the stent 900. The tie 90 may be coupled to, or otherwise associated with, major-axis end walls 927 of the frame. For example, the tie 970 may comprise a suture or other line tied to frame struts of the stent 900.

The stent 900 may be configured to be radially compressed to a low-profile delivery configuration, wherein upon deployment from a delivery system (e.g., catheter/sheath) utilized to deliver the implant 900, the stent 900 may self-expand, or may be balloon-expanded or otherwise mechanically expanded using certain instrumentation, wherein the expanded shape is constrained to the circular shape shown in FIG. 12B by a pre-coupled tie 970 that is coupled to the stent when the stent is delivered in the delivery system. Alternatively, the tic 970 may be coupled to the stent 900 after deployment of the stent 900. For example, the stent 900 may be deployed and assume a non-circular shape (e.g., oval), wherein the tie 970 may be coupled to the stent at the major-axis ends of the oval shape thereof, and/or tensioned to a desired reshaping tension, after expansion of the stent 900. The tie 970 may be pre-cut/configured to have the length dₜ when coupled to the stent 900, or the tie 970 may be tensioned in situ at the implantation site to achieve the desired circular shape of the stent 900.

The length dₜ of the sacrificial tie 970 spanning the diameter of the stent 900 may be greater than the biased minor-axis dₘᵢₙ₁ diameter of the non-circular stent 900, wherein the tension of the tie 970 across the inner diameter of the stent 900 causes the major-axis diameter of the stent 900 to contract to a reduced major-axis dimension dₘₐⱼ₂, as shown, which may be approximately equal to the effective length dₜ of the tie 970. Furthermore, the tension of the tie 970, by pulling-in the major-axis sidewalls 927 of the stent 900 inwardly, may cause the outward deflection of the sidewalls 925 in the direction of the minor axis of the stent 900, thereby increasing the minor axis dimension dₘᵢₙ of the stent 900 to approximate the length dₜ of the tie 970.

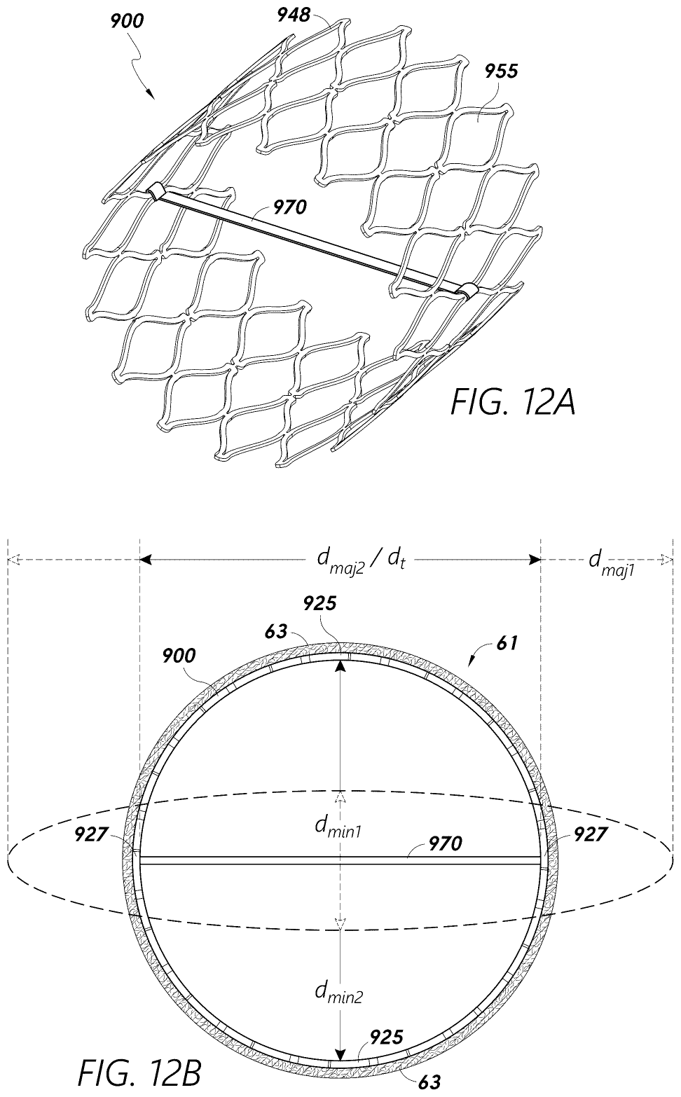

The support tie 970 may be configured to hold the circular shape of the stent 900 as shown in FIG. 12B for a period of time sufficient to allow tissue overgrowth 65 (see FIG. 13) to couple the stent 900 to the blood vessel 61. For example, by pulling the end walls 927 inward, thereby pushing the sidewalls 925 outward and against the walls 63 of the blood vessel 61, tissue overgrowth 65 may be permitted/facilitated between the walls 63 and the stent sidewalls 925, to thereby physically/mechanically couple/bind the sidewalls 925 to the blood vessel walls 63. The tissue growth 65 may project/accumulate through the open cells 955 of the stent 900, and may span across struts 948 of the stent frame on an internal diameter of the frame. In some implementations, the growth 65 may accumulate on a covering of the stent 900. After a sufficient period of time (e.g., eight hours or more, one or more days), the sacrificial support tie 970 may be removed or may dissolve of its own accord. For example, the support site 970 may comprise biodegradable material configured to at least partially dissolves and/or otherwise break-down over time, thereby defeating the circularizing structural support of the support tie 970.

FIG. 13 shows the non-circular stent 900 within the blood vessel 61 after the sacrificial support tie 970 has been dissolved, cut, and/or otherwise removed or de-tensioned and tissue overgrowth 65 has occurred, binding the sidewalls 925 of the stent 900 to the walls 63 of the blood vessel 61. As shown, after sufficient tissue overgrowth 65 has occurred, when the non-circular stent 900 reverts to its biased non-circular shape (e.g., oval-, peanut-shaped), the sidewalls 63 of the blood vessel may be inclined to deflect radially inwardly along with the sidewalls 925 of the stent 900, such that little or no gap is present between the sidewalls 925 and the blood vessel wall 63. In such configuration, cyclical reshaping of the stent 900 and blood vessel 61 between oval and circular shapes may occur in tandem. For example, in such configuration, major-axis elongation of the stent 900, as shown in FIG. 13, may cause radially-inward deflection of the sidewalls 925 thereof, which in-turn may pull the walls 63 of the blood vessel 61 radially inward to reshape the blood vessel 61 to a non-circular (e.g., oval) shape. Furthermore, as pressure increases in the blood vessel 61, such pressure forces may push outwardly on the blood vessel walls 63, the stent 900, and/or the tissue growth 65, wherein such forces may deflect the vessel walls 63, thereby pushing and/or pulling the stent walls 925 radially outwardly due to the tissue-coupling therewith, such that the reshaping of the blood vessel 61 to a more-circular shape in high-pressure conditions more efficiently reshapes the stent 900 to a more-circular shape (e.g., circular). Therefore, the tissue overgrowth 65 facilitated by the implementation of the support tie 970 can improve the efficiency and/or efficacy of the reshaping non-circular stent 900 for the purpose of promoting blood flow and/or enhancing compliance and/or perfusion characteristics associated with the target blood vessel 61.

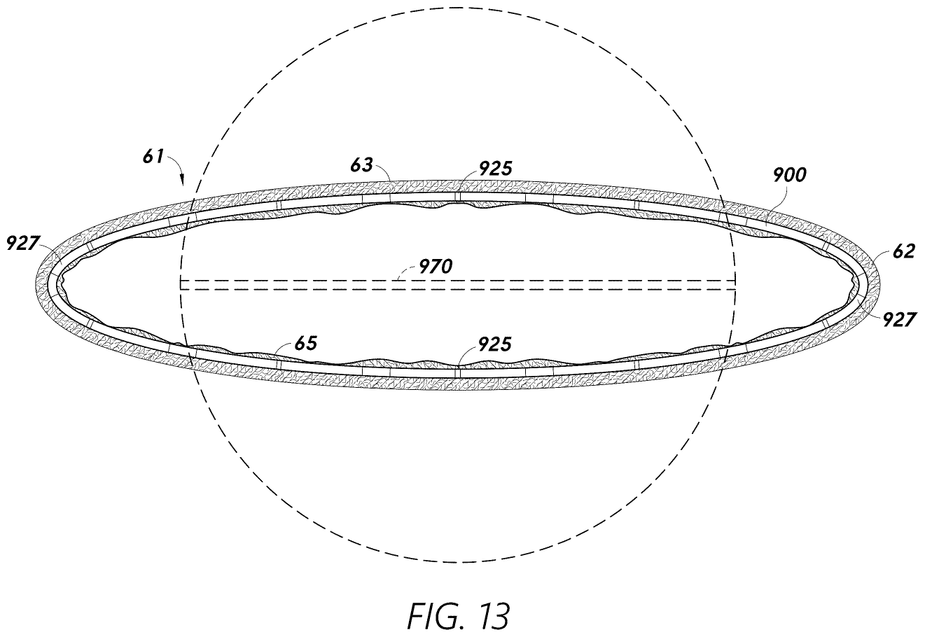

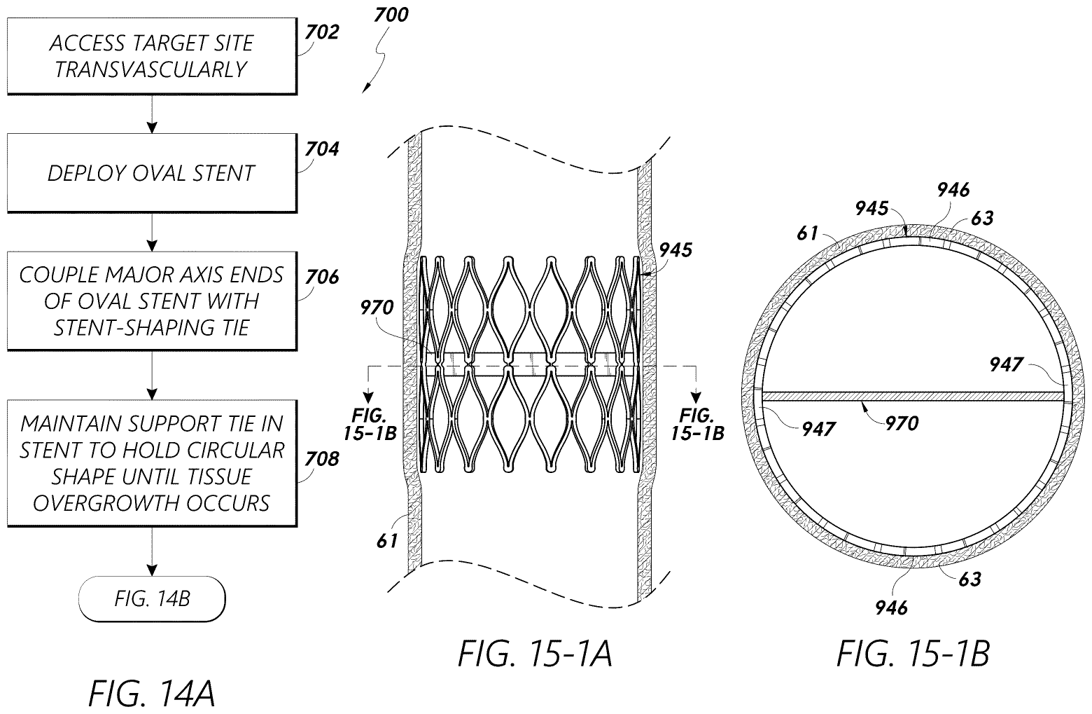

FIGS. 14A and 14B illustrate a flow diagram for a process 700 for coupling sidewalls of a non-circular stent 945 to blood vessel walls using a circularizing support tie 970 in accordance with one or more examples. FIGS. 15-1A. 15-1B, and 15-2 provide images of the stent 945, support tie 970, and certain anatomy corresponding to operations of the process 700 of FIGS. 14A and 14B according to one or more examples.

At block 702, the process 700 involves accessing a target segment of a blood vessel (e.g., aorta) using a transvascular path. For example, a delivery system may be advanced through a percutaneous introducer or other minimally-invasive access into the vasculature of the patient, and further within the vasculature to a target position within the aorta of the patient. As described above, the delivery system may include one or more catheters/sheaths or other feature configured to facilitate the advancement of the delivery system through the vasculature. The percutaneous entry may be at the femoral artery or other arterial blood vessel. The access to the target anatomy may be made with a delivery system configured to transport a non-circular stent 945 and one or more stent-circularizing support devices/structures, as described in detail herein.

The non-circular stent 945 may be transported to the target implantation site in a delivery configuration in which the frame of the stent is radially compressed. In some implementations, a circularizing support tie 970 is configured to be transported as coupled to the stent 945 and disposed at least partially within an inner diameter/lumen of the stent 945, which may allow for simultaneous deployment of the stent 945 and support structure/tie 970 and/or may otherwise simplify delivery of such devices. Alternatively, the stent 945 and support tie 970 may be delivered to the target anatomy in separate delivery systems, and/or in axially-offset positions within a common sheath or other delivery system component.

At block 704, the process 700 involves deploying the non-circular stent 945 (e.g., oval or peanut-shaped stent) from the delivery system, wherein the stent 945 is configured to assume a non-circular shape in a relaxed, non-pressurized state thereof. As referenced above, the stent 945 may be transported to the implantation site in a radially crimped/compressed configuration. The stent 945 may expand in accordance with self-expansion when deployed from the delivery system, or may be expanded using a balloon catheter or similar device.

At block 706, the process 700 involves coupling major-axis end portions of the stent with a stent-shaping tie 970 as described in detail herein to hold/force the stent 945 into a more-circular cross-sectional shape (compared to the relaxed, biased shape thereof), with respect to one or more lengthwise portions/segments of the stent 945. In some implementations, such coupling of the tie 970 and stent 945 can occur prior to delivery of the stent. That is, the tie 970 can be coupled to the stent when the stent is placed in the delivery system for delivery. In such implementations, the tie 970 may constrain the expansion of the stent in connection with block 704, such that the stent 945 expands to a circular shape rather than the biased non-circular (e.g., oval) shape of the stent 945. Alternatively, the operation(s) of block 706 may be performed after deployment of the stent 945, and may be performed using certain transcatheter instrumentation that may or may not be associated with the delivery system used to deploy the stent 945. For example, the tie 970 may be tied or otherwise coupled to the struts of the frame of the stent 945. In some implementations, the tic 970 is welded to the stent 945. In some implementations, the tie comprises a strut that is integrated with the frame of the stent 945.

The tie 970 may force the non-circular stent 945 into a circular shape, such as by forcing inward deflection of the major axis end walls 947 of the stent 945, thereby causing minor-axis sidewalls 946 to deflect radially outwardly. Both the stent 945 and the tie 970 may be deployed from the same delivery system simultaneously, with the tie 970 disposed within the stent 945, wherein expansion of the stent 945 may be achieved through self-expansion enabled by shape-memory characteristics of the stent 945, or through balloon expansion from within the axial channel of the stent 945. Where balloon expansion is implemented, and the tie 970 is pre-coupled to the stent, it may be desirable for the tie 970 to be elastic to allow for the stretching thereof to accommodate the expansion of the balloon, which may push the tie 970 against the inner diameter of the stent 945 and thereby cause a lengthening of the tie. Alternatively, the balloon expansion may be limited to axial segment(s) of the stent 945 that do not overlap with the tie 970. In view of the foregoing considerations, it may be desirable to implement the stent 945 as a self-expanding stent in examples in which the tie 970 is pre-coupled to the stent 945 to avoid interference between the tie and balloon catheter.

FIG. 15-1A shows the outer stent 945 and the inner support tie 970 deployed in the blood vessel 61, whereas FIG. 15-1B provides an axial cross-sectional detail of the stent 945 with the circularizing support tie 970 forcing the outer non-circular stent into a generally-circular shape, thereby approximating the sidewalls 946 of the stent 945 to adjacent walls 63 of the blood vessel 61 to facilitate tissue overgrowth to couple the stent walls 946 to the blood vessel walls 63 over time.

At block 708, the process 700 involves maintaining the support tie 970 (e.g., sacrificial, biodegradable) in position coupling the major-axis sides 947 of the stent 945 until tissue sufficient overgrowth 65 between the blood vessel walls 63 and the stent sidewalls 946 occurs. At block 710, the process 700 involves breaking, removing, dissolving, or otherwise decoupling the tic 970, and/or one or more portions thereof, from the major-axis end(s) 947 of the stent 945 to allow the stent 945 to transition to the biased, non-circular shape thereof. For example, the operation(s) associated with block 710 can involve cutting the tic 970 using instrumentation that may or may not be associated with the delivery system used to deploy the stent 945 and/or tie 970. Alternatively or additionally, the operation(s) associated with block 710 can involve allowing the tie 970 to dissolve, at least in part, such that the tension of the tie 710 spanning the inner diameter of the stent 945 is broken. For example, the tie 710, and/or one or more lengthwise or radial portions thereof, may comprise biodegradable material configured to dissolve in the presence of blood or other fluid after a period of time sufficient to facilitate tissue overgrowth. In some implementations, the support tie 970 may not fully dissolve, but rather certain portions or components thereof may dissolve or break in a manner as to reduce the ability of the tic 970 to force the circular shape of the non-circular stent 945, thereby allowing the stent 945 to break free from the forced circular state and assume a more natural, non-circular shape thereof (e.g., oval).

At block 712, the process 700 involves reshaping the blood vessel 16 to an oval or other non-circular cross-sectional shape using the stent 945. With the support tie 970 dissolved, the non-circular stent 945 may be permitted to return to its more natural configuration/shape, at least during periods of relatively low pressure within the blood vessel lumen. FIG. 15-2 shows the stent 945 having assumed a non-circular cross-sectional shape. Due to the tissue overgrowth, the sidewalls 63 of the blood vessel 61 may be adhered/coupled to the sidewalls 946 of the stent 945, such that the inward deflection of the sidewalls 946 pulls the blood vessel walls 63 inward in a commensurate manner, such that the stent 945 reshapes the blood vessel to a non-circular shape, as shown.

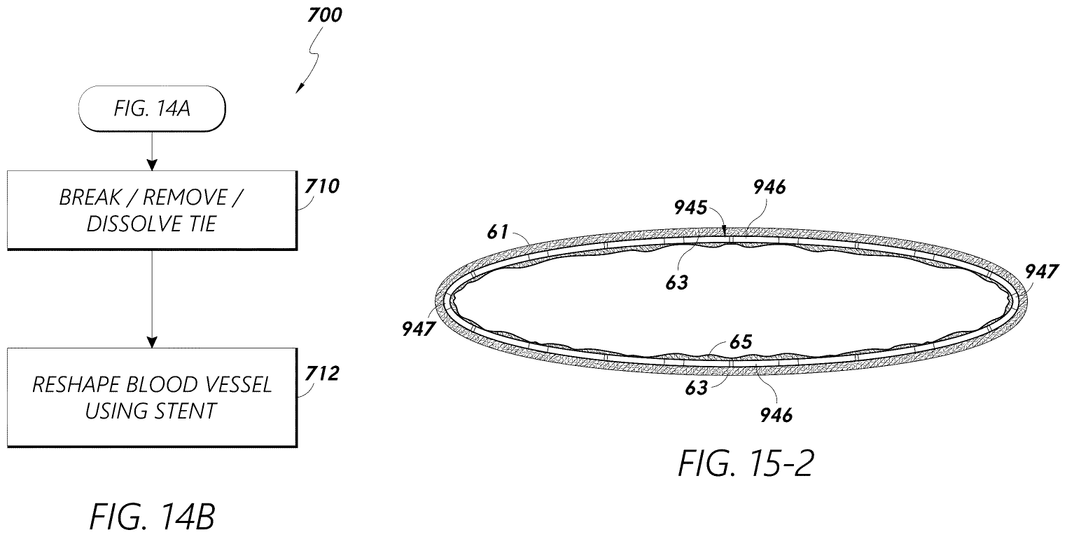

FIG. 16 shows an axial view of a non-circular stent 1600 having inwardly-deflected sidewalls 1625 in accordance with one or more examples. The stent 1600 may represent an example implementation of any of the non-circular/oval stents disclosed herein, or portion(s) thereof. The stent 1600 forms/defines an elongate/elongated tubular member, which forms a blood flow lumen 1649.

The stent 1600 may have an hourglass and/or peanut (e.g., non-circular) shape with respect to the axial cross-section shown in FIG. 16. The stent 1600 may have a major axis diameter/dimension that is greater than a minor axis diameter/dimension of the oval cross-section.

In some examples, the stent 1600 may be biased to a shape having a minor axis dimension that is non-constant along the major axis dimension, which forms an externally-concave/internally-convex surface/form with respect to the minor axis sidewalls 1625. For example, the minor axis sidewalls 1625 may have a diameter dimension at a center thereof (with respect to the major axis dimension) that is less than the diameter/dimension at/towards the end portions of the minor-axis sidewalls 1625. The inwardly-deflected walls 1625 can allow for a transition reshaping between peanut-shaped, to outwardly-bowed oval shape, and ultimately to circular/more-circular shape.

The stent 1600 may be biased (e.g., memory-set using shape-memory materials, which can include nitinol) toward a peanut and/or similar shape. With the sidewalls 1625 deflected inwardly, as shown, the stent frame 1631 may form bulging/bulbous portions 1669a, 1669b (e.g., ends) on either side (e.g., opposing sides) of the minor axis, wherein such portions can have a diameter/dimension that is greater than the shortened diameter. The sidewalls 1625 may comprise a midsection disposed between the bulbous portions 1669. In some examples, the covering 1630 may extend along a full surface of the midsection. The bulbous portions 1669 may have generally circular and/or semi-circular forms and/or shapes.

In some examples, the stent 1600 may be configured to be implanted in a blood vessel (e.g., aorta). The stent 1600 can comprise one or more circularizing tether/tie features (see, e.g., FIGS. 12A and 12B) configured to extend from and/or between the bulbous portions 1669a, 1669b and/or side walls 1625. The circularizing features can include one or more biodegradable sutures configured to encourage endothelization around the frame. For example, the circularizing features can be configured to facilitate and/or maintain a generally circular form of the stent 1600 immediately after implantation to avoid gaps forming between the stent 1600 and the blood vessel walls.

In some examples, the bulbous portions 1669 may not comprise a covering 1630. The bulbous portions 1669 may be configured to be aligned with one or more branching blood vessels (e.g., intercostal arteries) and/or may be configured to allow blood flow through the frame 1631 and/or into branching blood vessels. In some examples, one or more bulbous portions 1669 (e.g., the first bulbous portion 1669a) may comprise a coating configured to inhibit ingrowth of tissue around the bulbous portion(s) 1669.

The side walls 1625 may be configured transition between peanut-shaped and circle-shaped during diastole and systole and/or in response to attachment and/or removal of one or more circularizing features. In some examples, the bulbous portions 1669 may be configured to remain substantially unchanged in shape during diastole, systole, and/or attachment and/or removal of the circularizing features. Accordingly, to cause shape changes of a blood vessel, the side walls 1625 may advantageously be configured to securely attach to the blood vessel walls.

In some examples, the stent 1600 may comprise (at least at delivery) one or more ties, sutures, stents, and/or other sacrificial features configured to be disposed at least partially within the inner lumen 1649 of the stent 1600 and/or configured to force the frame 1631 to a generally circular shape and/or a shape that is more circular than the biased non-circular shape of the stent 1600. Such features can be biodegradable and/or may comprise one or more biodegradable materials. In some examples, the frame 1731 and the sacrificial features may be radially compressible. The frame 1631 and/or the sacrificial features may be configured to be transported and/or delivered together in a delivery system (e.g., catheter) in which the frame 1631 is radially compressed and/or the sacrificial feature(s) may be radially compressed and/or disposed within the radially compressed frame 1631. A sacrificial feature (e.g., tie) may have a diameter that is greater than the minor-axis dimension of the frame 1631 and/or that is smaller than the major-axis dimension of the frame 1631.

In some examples, only the side walls 1625 of the stent 1600 may comprise a covering 1630. The covering 1630 may be attached to and/or configured to extend along and/or enclose an outer surface and/or inner surface of the frame 1631 at the side walls 1625. In some examples, the covering 1630 may attach to and/or enclose only a first portion of the stent 1600 and/or may not attach to and/or enclose a second portion of the stent 1600. For examples, the covering 1630 may not extend along the bulbous portions 1669. The covering 1630 may comprise various biodegradable materials and/or may be configured to promote endothelization at one or more portions of the stent 1600. For example, the covering 1630 may at least partially enclose the side walls 1625 to promote endothelization around the side walls 1625 to facilitate attachment and/or shape change translation between the side walls 1625 and the blood vessel walls.

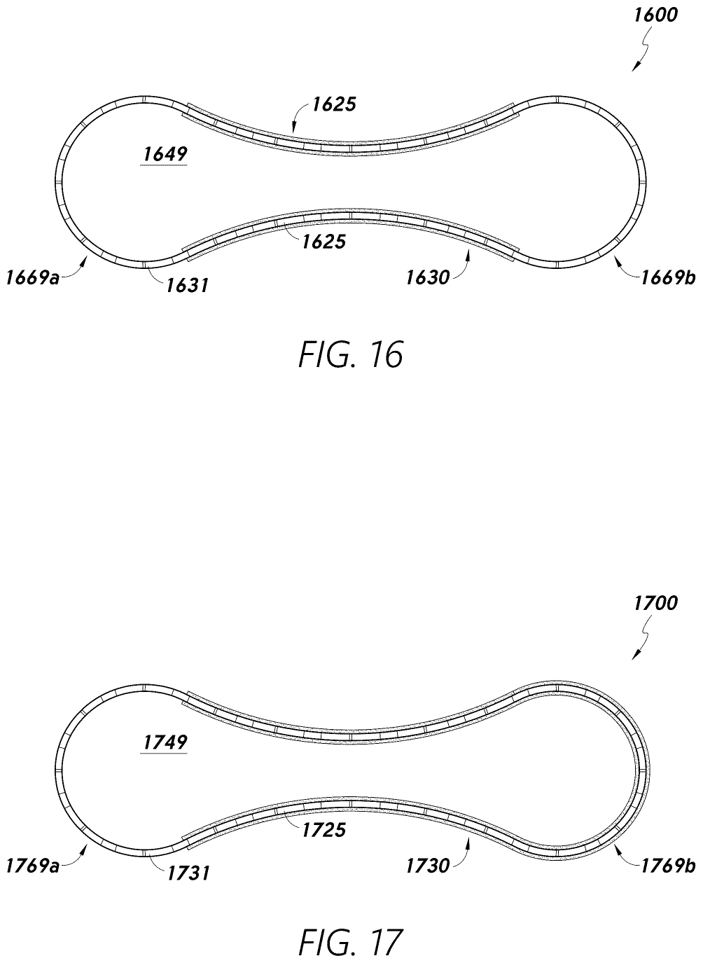

FIG. 17 shows an axial view of a non-circular stent 1700 having inwardly-deflected sidewalls 1725 in accordance with one or more examples. The stent 1700 may represent an example implementation of any of the non-circular/oval stents disclosed herein, or portion(s) thereof. The stent 1700 forms/defines an elongate/elongated tubular member, which forms a blood flow lumen 1749.

The stent 1700 may have an hourglass and/or peanut shape with respect to the axial cross-section shown in FIG. 17. The stent 1700 may have a major axis diameter/dimension that is greater than a minor axis diameter/dimension of the oval cross-section.

In some examples, the stent 1700 may be biased to a shape having a minor axis dimension that is non-constant along the major axis dimension, which forms an externally-concave/internally-convex surface/form with respect to the minor axis sidewalls 1725. For example, the minor axis sidewalls 1725 may have a diameter dimension at a center thereof (with respect to the major axis dimension) that is less than the diameter/dimension at/towards the end portions of the minor-axis sidewalls 1725. The inwardly-deflected walls 1725 can allow for a transition reshaping between peanut-shaped, to outwardly-bowed oval shape, and ultimately to circular/more-circular shape.

The stent 1700 may be biased (e.g., memory-set using shape-memory materials, which can include nitinol) toward a peanut and/or similar shape. With the sidewalls 1725 deflected inwardly, as shown, the stent frame 1731 may form bulging/bulbous portions 1769a, 1769b on either side of the minor axis, wherein such portions can have a diameter/dimension that is greater than the shortened diameter.

In some examples, the stent 1700 may be configured to be implanted in a blood vessel (e.g., aorta). The stent 1700 can comprise one or more circularizing tether/tie features (see, e.g., FIGS. 12A and 12B) configured to extend from and/or between the bulbous portions 1769a, 1769b and/or side walls 1725. The circularizing features can include one or more biodegradable sutures configured to encourage endothelization around the frame. For example, the circularizing features can be configured to facilitate and/or maintain a generally circular form of the stent 1700 immediately after implantation to avoid gaps forming between the stent 1700 and the blood vessel walls.

In some examples, the bulbous portions 1769 may not comprise a covering 1730. The bulbous portions 1769 may be configured to be aligned with one or more branching blood vessels (e.g., intercostal arteries) and/or may be configured to allow blood flow through the frame 1731 and/or into branching blood vessels.

The side walls 1725 may be configured transition between peanut-shaped and circle-shaped during diastole and systole and/or in response to attachment and/or removal of one or more circularizing features. In some examples, the bulbous portions 1769 may be configured to remain substantially unchanged in shape during diastole, systole, and/or attachment and/or removal of the circularizing features. Accordingly, to cause shape changes of a blood vessel, the side walls 1725 may advantageously be configured to securely attach to the blood vessel walls.

In some examples, only the side walls 1725 of the stent 1700 may comprise a covering 1730. The covering 1730 may be configured to extend along and/or enclose an outer surface and/or inner surface of the frame 1731 at the side walls 1725. The covering 1730 may not extend along the bulbous portions 1769. The covering 1730 may comprise various biodegradable materials and/or may be configured to promote endothelization at one or more portions of the stent 1700. For example, the covering 1730 may at least partially enclose the side walls 1725 to promote endothelization around the side walls 1725 to facilitate attachment and/or shape change translation between the side walls 1725 and the blood vessel walls. In some examples, the covering 1730 may be at least partially biodegradable.

In some examples, the covering 1730 may extend along and/or enclose one or more bulbous portions 1769. For example, as shown in FIG. 17, the covering 1730 may at least partially enclose a second bulbous portion 1769b. The covering 1730 may extend along the side walls 1725 (e.g., midsection) and/or continuously extend to the second bulbous portion 1769b. In some examples, the stent 1700 may be placed in a blood vessel such that the second bulbous portion 1769b may not be disposed at and/or in contact with one or more branching blood vessels. A first bulbous portion 1769a may be configured to be disposed at and/or in contact with one or more branching blood vessels and/or may not be enclosed by the covering 1730, as shown in FIG. 17. For example, the covering 1730 may end along the side walls 1725 and/or partially along the first bulbous portion 1769a. Accordingly, the first bulbous portion 1769a may be at least partially uncovered and/or may be configured to allow blood flow from the central lumen 1749 out of the stent 1700 and/or from outside the stent 1700 into the central lumen 1749.

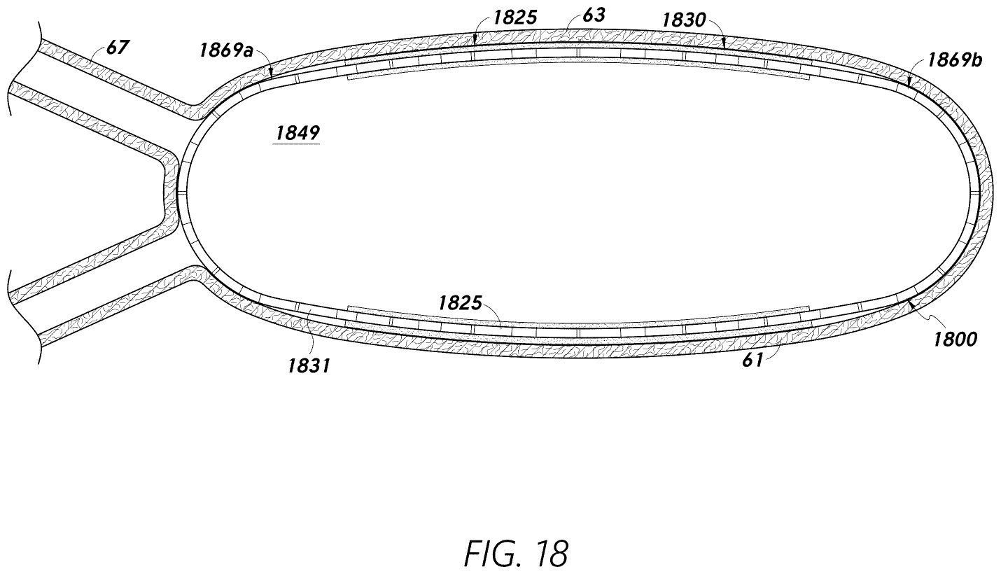

FIG. 18 shows an axial view of a peanut-shaped stent 1800 deployed within a blood vessel 61 in accordance with one or more examples. Once deployed within a blood vessel 61, during diastolic pressure, the stent sidewalls 1825 may remain at least partially inwardly deflected or, due to blood pressure levels present (even during low-pressure conditions, e.g., diastole), the stent 1800 and/or blood vessel 61 may deform into a substantially oval, non-peanut shape. When subjected to the higher pressures of systole, the stent 1800 and/or blood vessel 61 may deform to a more circular shape. The stent 1800 forms/defines an elongate/elongated tubular member, which forms a blood flow lumen 1849.

The minor-axis sidewalls 1825 may have a default inward deflection (e.g., forming a peanut shape). Thus, the stent 1800 may form gaps between the sidewalls 1825 and the blood vessel sidewalls 63 when the stent 1800 is initially deployed within the blood vessel 61. That is, the tendency of the stent sidewalls 1825 to pull away from the blood vessel sidewalls 63 may be relatively high, and therefore, without tissue overgrowth coupling the vessel walls 63 to the stent walls 1825, the reshaping ability/function of the stent 1800 may be at least partially impaired/impeded. Furthermore, where the gaps are present between the stent walls 1825 and the vessel walls 63, blood may collect and stagnate in such spaces, presenting embolism risk and/or impeding outward deflection of the stent walls 1825 to circularize during the systolic phase of the cardiac cycle. Therefore, facilitating tissue overgrowth of the stent walls 1825 may be of particular significance with respect to peanut-shaped stents.

In some examples, the stent 1800 can include one or more biodegradable sutures attached between the side walls 1825 and/or bulbous portions 1869 to force a circular configuration for a certain period of time, during which at least the side walls 1825 may be endothelized. For example, the side walls 1825 can comprise a dissolvable covering 1830 configured to promote endothelization. After an amount of time allowing tissue growth attaching the elongated side walls 1825 to the wall(s) of the vessel 63, the sutures and/or covering 1830 may be configured to dissolve. The stent 1800 may then be free to assume the unbiased peanut-shaped configuration shown in FIG. 18 during diastole. As shown in FIG. 18, endothelization may cause the walls 63 of the vessel 61 to be pulled along with the stent 1800 towards an inwardly-deflected and/or peanut shape. In some cases, resistance from the blood vessel walls 63 may prevent the vessel 61 and/or stent 1800 from reaching an inwardly-deflected and/or peanut shape. However, the bias of the stent 1800 may cause the vessel 61 to form a generally oval shape (e.g., during diastole). The stent 1800 may be configured to transition to a circular shape during systole, forcing the wall(s) of the vessel 63 (e.g., at least the portions of the wall(s) attached to side walls 1825 and/or movable portions of the frame 1831) to move therewith.

The stent 1800 can be implanted such that at least one of one or more bulbous portions 1869 may be aligned against one or more branching blood vessels 67. The bulbous portions 1869 may have circular and/or semi-circular forms and/or may be disposed on either side of the side walls 1825. In some examples, one or more coverings 1830 of the stent 1800 may not extend along one or more bulbous portions 1869 (e.g., may not extend along at least a first bulbous portion 1869a and/or may extend along a second bulbous portion 1869b). Blood may be enabled to freely flow through the inner flow channel 1849 of the stent 1800, through the exposed portion of the frame 1831 along the bulbous portions 1869 (e.g., at least a first bulbous portion 1869a) into the branching vessel(s) 67. In some examples, a second bulbous portion 1869b not contact one or more branching vessels 67.

In order to promote tissue ingrowth between the side walls 1825 of the stent 1800 and/or various non-circular (e.g., oval, peanut-shaped) stents of the present disclosure and blood vessel walls, certain support structures/devices can be implemented to force the shape of a non-circular-biased stent 1800 to a cross-sectional shape that is more circular than the biased/relaxed shape thereof, such that the stent walls 1825 are more evenly/consistently in contact with and/or proximate to the walls of the target blood vessel for a sufficient period of time to allow for the desired tissue overgrowth to occur. Such stent-shaping/circularizing supports can be configured to reshape the stent 1800 for only a transitory/temporary period of time sufficient to allow for the desired tissue overgrowth to occur, after which point the support(s) may dissolve or otherwise be removed from connection/contact with the non-circular stent 1800 to allow the stent 1800 to return towards a biased non-circular shape thereof.

Tissue overgrowth of a non-circular stent 1800 that is forced to a more-circular shape by support structure(s) of the present disclosure can involve endothelialization of the blood vessel wall over the stent frame 1831 and/or covering 1830 associated therewith. Generally, the deployment of the stent 1800 in the target blood vessel 63 can cause injury to the vascular wall and endothelium, which comprises a layer of endothelial cells lining the vascular wall that promotes homeostasis. The injury to the blood vessel wall can result in inflammation, repair, and the development of neointimal hyperplasia. The ability of the endothelium to repair itself can depend on the migration of surrounding mature endothelial cells, and the attraction and adhesion of circulating endothelial progenitor cells (EPCs) to the area where the stent is deployed, forming overgrowth of endothelial-like cells.

In some examples, stent frames 1831 and/or coverings 1830 can be implemented with drug-eluting features/coatings that at least partially interrupt the natural tissue overgrowth process. Alternatively, stents 1800 can be implemented with treatments that accelerate the reendothelialization of the damaged arterial segment following stent deployment. Such tissue-growth promotion can reduce risks of neointimal hyperplasia and stent thrombosis. In order to promote the desired coupling between stent walls 1825 and blood vessel walls to facilitate blood vessel reshaping as described in detail herein, stent frames and/or coverings of stents of the present disclosure can be treated with agents that augment the generation and/or accumulation of endothelial cells or EPCs in the area of the deployed stent. In some implementations, the stent frame 1831 and/or covering 1830 can be seeded with endothelial cells or EPCs. For example, a stent 1800 can be coated with anti-CD34 antibodies to attract endothelial/EPC cells to the stent. The stent-reshaping/circularizing structures disclosed herein can be designed to hold the associated stent in a relatively circular shape for a period of time that correlates with the expected tissue overgrowth period based on the tissue-growth-promoting/inhibiting features of the stent implant. Bulbous portions 1869 and/or various alternate sides of the stent 1800 may be uncovered to allow blood flow through such portions.

Stent-circularizing support structures of the present disclosure can advantageously be biodegradable, such that after a period of time, such structures cease forcing a more-circular shape of the stent, thereby allowing the stent to assume/revert-to a biased non-circular shape to thereby reshape the target blood vessel. Such reshaping of the target blood vessel advantageously occurs after the support structure has held the stent in a relatively circular shape to promote and allow for tissue ingrowth and coupling between the stent and the blood vessel. Stent-circularizing supports of the present disclosure can take the form of stents, ties/tethers, or other forms.

### Additional Examples

Depending on the example, certain acts, events, or functions of any of the processes or algorithms described herein can be performed in a different sequence, may be added, merged, or left out altogether. Thus, in certain examples, not all described acts or events are necessary for the practice of the processes.

Provided below is a list of examples, each of which may include aspects of any of the other examples disclosed herein. Furthermore, aspects of any example described above may be implemented in any of the numbered examples provided below.

Example 1: An implant device comprising a first stent having a biased non-circular axial cross-sectional shape, and a second stent having a biased circular axial cross-sectional shape, the second stent being configured to be disposed within an inner channel of the first stent and to force the first stent to a shape that is more circular than the biased non-circular shape.

Example 2: The implant device of any example herein, in particular example 1, wherein the second stent is biodegradable.

Example 3: The implant device of any example herein, in particular example 1 or example 2, wherein the second stent comprises biodegradable material.

Example 4: The implant device of any example herein, in particular any of examples 1-3, wherein the first stent and the second stent are radially compressible.

Example 5: The implant device of any example herein, in particular any of examples 1-4, wherein the first stent and the second stent are configured to be transported together in a delivery system in which the first stent is radially compressed and the second stent is radially compressed and disposed within the radially compressed first stent.

Example 6: The implant device of any example herein, in particular any of examples 1-5, wherein the biased non-circular shape of the first stent has a major-axis dimension and a minor-axis dimension, the major-axis dimension being greater than the minor-axis dimension.

Example 7: The implant device of any example herein, in particular example 6, wherein the second stent has a diameter that is greater than the minor-axis dimension of the first stent and smaller than the major-axis dimension of the first stent.

Example 8: The implant device of any example herein, in particular example 6 or example 7, wherein the biased non-circular shape is an oval shape.

Example 9: The implant device of any example herein, in particular any of examples 6-8, wherein the biased non-circular shape is a peanut shape.

Example 10: An implant device comprising a stent frame having a circular shape, wherein the stent frame has sufficient rigidity to reshape a non-circular stent in which the stent frame is deployed to a more-circular shape, and the stent frame is at least partially biodegradable.

Example 11: The implant device of any example herein, in particular example 10, wherein the stent frame is radially compressible.

Example 12: The implant device of any example herein, in particular example 10 or example 11, wherein the stent frame has a diameter between 1-3 cm.

Example 13: The implant device of any example herein, in particular any of examples 10-12, wherein the stent frame is configured to biodegrade, at least in part, after a sufficient time for tissue overgrowth to occur coupling a stent in which the stent frame is deployed to a blood vessel in which the stent is deployed.

Example 14: The implant device of any example herein, in particular any of examples 10-13, wherein the stent frame is configured to biodegrade after a period of at least four days.

Example 15: The implant device of any example herein, in particular example 14, wherein the stent frame is configured to biodegrade after a period of at least seven days.

Example 16: The implant device of any example herein, in particular example 15, wherein the stent frame is configured to biodegrade after a period of at least twenty-eight days.

Example 17: A method of coupling a stent to a blood vessel, the method comprising deploying a first stent in a target blood vessel segment, the first stent having a non-circular relaxed shape, forcing the first stent to a more-circular shape compared to the non-circular relaxed shape using a second stent disposed in an inner channel of the first stent, maintaining the second stent within the first stent for a period of time sufficient to allow tissue overgrowth to couple the first stent to a wall of the target blood vessel segment, and removing the second stent to allow the first stent to reshape the target blood vessel segment to a non-circular shape.

Example 18: The method of any example herein, in particular example 17, wherein said deploying the first stent comprises expanding the first stent from a radially-compressed delivery configuration to an expanded configuration.

Example 19: The method of any example herein, in particular example 18, wherein said forcing the first stent to the more-circular shape comprises expanding the second stent within the inner channel of the first stent.

Example 20: The method of any example herein, in particular example 19, wherein said expanding the second stent is performed using a balloon device disposed within a channel of the second stent.

Example 21: The method of any example herein, in particular example 19 or example 20, wherein said expanding the second stent causes said expanding the first stent.

Example 22: The method of any of any example herein, in particular examples 19-21, wherein said expanding the second stent is performed using shape-memory features of the second stent.

Example 23: The method of any example herein, in particular example 17 or example 18, wherein said removing the second stent comprises retrieving the second stent using a transcatheter device.

Example 24: The method of any example herein, in particular example 17 or example 18, wherein said removing the second stent comprises dissolving at least a portion of the second stent.

Example 25: The method of any example herein, in particular any of examples 17-24, wherein said removing the second stent comprises maintaining the second stent within the first stent for a tissue growth period to allow the second stent to at least partially dissolve.

Example 26: The method of any example herein, in particular example 25, wherein the tissue growth period is at least one day.

Example 27: The method of any example herein, in particular any of examples 17-26, wherein the second stent is physically coupled to the first stent.

Example 28: The method of any example herein, in particular any of examples 17-27, further comprising advancing the first stent and the second stent to the target blood vessel segment in a transcatheter delivery system.

Example 29: The method of any example herein, in particular example 28, wherein the second stent is disposed within an inner channel of the first stent when the first stent and the second stent are in the transcatheter delivery system.

Example 30: An implant device comprising a stent having a biased non-circular axial cross-sectional shape, and a tie coupled to major-axis sides of the stent and holding the major-axis sides of the stent in a manner as to force a more-circular shape of the stent relative to the biased non-circular shape of the stent.

Example 31: The implant device of any example herein, in particular example 30, wherein the tie is biodegradable.

Example 32: The implant device of any example herein, in particular example 30 or example 31, wherein the tie comprises biodegradable material.

Example 33: The implant device of any example herein, in particular any of examples 30-32, wherein the stent and the tie are configured to be transported together in a delivery system in which the stent is radially compressed and the tie is disposed at least partially within the radially compressed stent.

Example 34: The implant device of any example herein, in particular any of examples 30-33, wherein the biased non-circular shape of the stent has a major-axis dimension and a minor-axis dimension, the major-axis dimension being greater than the minor-axis dimension.

Example 35: The implant device of any example herein, in particular example 34, wherein the tie has a length that is greater than the minor-axis dimension of the stent and smaller than the major-axis dimension of the stent.

Example 36: The implant device of any example herein, in particular example 34 or example 35, wherein the biased non-circular shape is an oval shape.

Example 37: The implant device of any example herein, in particular examples 34-36, wherein the biased non-circular shape is a peanut shape.

Example 38: The implant device of any example herein, in particular any of examples 30-37, wherein the tie is integrated with a frame of the stent.

Example 39: The implant device of any example herein, in particular any of examples 30-38, wherein the tie is tied to opposing walls of a frame of the stent.

Example 40: An implant device comprising a biodegradable tie, wherein the biodegradable tie is configured to be coupled to diametrically-opposite walls of a non-circular stent and tensioned to reshape the non-circular stent to a more-circular shape.

Example 41: The implant device of any example herein, in particular example 40, wherein the tic has a length between 1-3 cm.

Example 42: The implant device of any example herein, in particular example 40 or example 41, wherein the tie is configured to biodegrade, at least in part, after a sufficient time for tissue overgrowth to occur over at least portions of a stent to which the tie is coupled.

Example 43: The implant device of any example herein, in particular any of examples 40-42, wherein the tie is configured to biodegrade after a period of at least four days.

Example 44: The implant device of any example herein, in particular any of examples 40-43, wherein the tie is configured to biodegrade after a period of at least twenty-eight days.

Example 45: A method of coupling a stent to a blood vessel, the method comprising deploying a stent in a target blood vessel segment, the stent having a non-circular biased shape, forcing the stent to a more-circular shape compared to the non-circular biased shape using a tie coupled across an inner diameter of the stent in a tensioned state, maintaining the tie in the tensioned state for a period of time sufficient to allow tissue overgrowth to couple the stent to a wall of the target blood vessel segment, and de-tensioning the tie to allow the stent to reshape the blood vessel segment to a non-circular shape.

Example 46: The method of any example herein, in particular example 45, wherein said deploying the stent comprises expanding the stent from a radially-compressed delivery configuration to an expanded configuration.

Example 47: The method of any example herein, in particular example 46, wherein said forcing the first stent to the more-circular shape comprises tensioning the tie.

Example 48: The method of any example herein, in particular any of examples 45-47, wherein said de-tensioning the tie comprises retrieving the tie using a transcatheter device.

Example 49: The method of any example herein, in particular any of examples 45-48, wherein said de-tensioning the tie comprises cutting the tie using a transcatheter device.

Example 50: The method of any example herein, in particular any of examples 45-49, wherein said de-tensioning the tie comprises dissolving at least a portion of the tie.

Example 51: The method of any example herein, in particular any of examples 45-50, wherein said de-tensioning the tie comprises maintaining the tie in the tensioned state for a tissue growth period to allow the tie to at least partially dissolve.

Example 52: The method of any example herein, in particular example 51, wherein the tissue growth period is at least one day.

Example 53: The method of any example herein, in particular any of examples 45-52, wherein the tie is coupled across the inner diameter of the stent prior to said deploying the stent.

Example 54: The method of any example herein, in particular any of examples 45-53, further comprising coupling the tie to the stent subsequent to said deploying the stent.

Example 55: The method of any example herein, in particular any of examples 45-54, wherein, when the tie is coupled across the inner diameter of the stent, end portions of the tie are tied to respective strut frames of the stent.

Example 56: The method of any example herein, in particular any of examples 45-55, further comprising advancing the stent and the tie to the target blood vessel segment in a transcatheter delivery system.

Example 57: The method of any example herein, in particular example 56, wherein the tie is coupled to the stent and disposed at least partially within an inner channel of the stent when the stent and the tie are in the transcatheter delivery system.

Example 58: An implant device of any example herein including: a frame having a biased non-circular axial cross-sectional shape; and a covering attached to the frame and enclosing a first portion of the frame, wherein a second portion of the frame is not enclosed by the covering.

Example 59: The implant device of any example herein, further including a tie configured to be disposed within an inner channel of the frame and to force the frame to a shape that is more circular than the biased non-circular shape.

Example 60: The implant device of any example herein, wherein the tie is biodegradable.

Example 61: The implant device of any example herein, wherein the tie includes biodegradable material.

Example 62: The implant device of any example herein, wherein the frame and the tie are radially compressible.

Example 63: The implant device of any example herein, wherein the frame and the tie are configured to be transported together in a delivery system in which the frame is radially compressed and the tie is radially compressed and disposed within the radially compressed frame.

Example 64: The implant device of any example herein, wherein the biased non-circular shape of the frame has a major-axis dimension and a minor-axis dimension, the major-axis dimension being greater than the minor-axis dimension.

Example 65: The implant device of any example herein, wherein the tie has a diameter that is greater than the minor-axis dimension of the frame and smaller than the major-axis dimension of the frame.

Example 66: The implant device of any example herein, wherein the biased non-circular shape is an oval shape.

Example 67: The implant device of any example herein, wherein the biased non-circular shape is a peanut shape.

Example 68: The implant device of any example herein, wherein the frame includes generally circular bulbous portions on opposing ends of the frame, and wherein the frame includes a midsection between the bulbous portions.

Example 69: The implant device of any example herein, wherein the covering encloses the midsection.

Example 70: The implant device of any example herein, wherein the covering encloses at least one of the bulbous portions.

Example 71: The implant device of any example herein, wherein the covering does not enclose at least one of the bulbous portions.

Example 72: The implant device of any example herein, wherein the midsection includes inwardly-deflected side walls.

Example 73: The implant device of any example herein, wherein at least one of the bulbous portions is configured to be aligned with one or more branching blood vessels.

Example 74: The implant device of any example herein, wherein the covering is biodegradable.

Example 75: A method of coupling a stent to a blood vessel, the method including: deploying a stent in a target blood vessel segment, the stent having a non-circular relaxed shape and including a covering enclosing at least a portion of the stent; and aligning a first end of the stent with one or more branching blood vessels that branch from the target blood vessel segment, wherein the first end is not enclosed by the covering.

Example 76: The method of any example herein, further including: forcing the stent to a more-circular shape compared to the non-circular relaxed shape using a tie disposed in an inner channel of the stent; maintaining the tie within the stent for a period of time sufficient to allow tissue overgrowth to couple the stent to a wall of the target blood vessel segment; and removing the tie to allow the stent to reshape the target blood vessel segment to a non-circular shape.

Example 77: The method of any example herein, wherein the covering is configured to cause in-growth of tissue.

Conditional language used herein, such as, among others, “can,” “could,” “might.” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including.” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example. Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of coupling a stent to a blood vessel, the method comprising:
deploying a stent in a target blood vessel segment, the stent having a non-circular biased shape;
forcing the stent to a more-circular shape compared to the non-circular biased shape using a tie coupled across an inner diameter of the stent in a tensioned state;
maintaining the tie in the tensioned state for a period of time sufficient to allow tissue overgrowth to couple the stent to a wall of the target blood vessel segment; and
de-tensioning the tie to allow the stent to reshape the target blood vessel segment to a non-circular shape.

2. The method of claim 1, wherein said deploying the stent comprises expanding the stent from a radially-compressed delivery configuration to an expanded configuration.

3. The method of claim 2, wherein said forcing the stent to the more-circular shape comprises tensioning the tie.

4. The method of claim 1, wherein said de-tensioning the tie comprises retrieving the tie using a transcatheter device.

5. The method of claim 1, wherein said de-tensioning the tie comprises cutting the tie using a transcatheter device.

6. The method of claim 1, wherein said de-tensioning the tie comprises dissolving at least a portion of the tie.

7. The method of claim 1, wherein said de-tensioning the tie comprises maintaining the tie in the tensioned state for a tissue growth period to allow the tie to at least partially dissolve from exposure to blood within the target blood vessel segment.

8. The method of claim 7, wherein the tissue growth period is at least one day.

9. A method of coupling a stent to a blood vessel, the method comprising:
providing a stent implant having a non-circular biased shape; and
securing a biodegradable structure to an inner diameter of the stent implant, such that the biodegradable structure, as secured, forces the stent implant to a more-circular shape compared to the non-circular biased shape when the stent implant is in an expanded configuration.

10. The method of claim 9, further comprising:
deploying the stent implant within a target blood vessel segment;
expanding the stent implant within the target blood vessel segment;
maintaining the expanded stent implant in the more-circular shape within the target blood vessel segment using the biodegradable structure; and
causing the stent implant to revert to a less-circular shape by allowing the biodegradable structure to at least partially dissolve within the target blood vessel segment.

11. The method of claim 9, further comprising:
compressing the stent implant to a radially-compressed delivery configuration with the biodegradable structure secured within a lumen of the stent implant;
placing the compressed stent implant in a delivery sheath;
advancing the delivery sheath to a target location within a blood vessel via an intravascular path;
deploying the stent implant and the biodegradable structure from the delivery sheath within the blood vessel; and
expanding the stent implant and the biodegradable structure in a manner that forces the stent implant to a circular cross-sectional shape due to physical engagement of the biodegradable structure with the stent implant.

12. The method of claim 9, further comprising:
compressing the stent implant to a radially-compressed delivery configuration;
placing the compressed stent implant in a delivery sheath;
advancing the delivery sheath to a target location within a blood vessel via an intravascular path;
deploying the stent implant from the delivery sheath within the blood vessel;
expanding the stent implant in a manner that causes the stent implant to assume a non-circular shape within the blood vessel;
intravascularly advancing the biodegradable structure to a position within a lumen of the stent implant within the blood vessel; and
securing the biodegradable structure to the stent implant from within the lumen of the stent implant within the blood vessel, thereby causing the stent implant to be re-shaped to a more-circular shape within the blood vessel.

13. The method of claim 12, wherein said intravascularly advancing the biodegradable structure to the position within the lumen of the stent implant within the blood vessel is performed using a delivery device that is separate from the delivery sheath used to transport the stent implant.

14. The method of claim 12, wherein:
the biodegradable structure comprises a biodegradable scaffold formed of a plurality of connected struts and having a circular expanded cross-sectional shape; and
said securing the biodegradable structure to the stent implant from within the lumen of the stent involves expanding the biodegradable scaffold within the lumen of the stent implant to force the stent implant to the more-circular shape.

15. The method of claim 14, wherein the biodegradable scaffold, prior to dissolving, has a greater rigidity than the stent implant.

16. The method of claim 12, wherein:
the biodegradable structure comprises a biodegradable tie; and
said securing the biodegradable structure to the stent implant from within the lumen of the stent implant involves tying first and second portions of the biodegradable tie to respective struts of the stent implant from within the lumen of the stent implant.

17. The method of claim 16, further comprising tensioning the tie to force the stent implant to the more-circular shape.

18. The method of claim 16, wherein the first and second portion of the biodegradable tie are tied to respective major-axis sidewalls of the stent implant.

19. The method of claim 9, wherein:
the biodegradable structure comprises a tie;
said securing the biodegradable structure to the inner diameter of the stent implant involves tying first and second ends of the tie to struts of the stent implant on diametrically opposite areas of the stent implant, such that the tie spans a lumen of the stent implant along a central diameter; and
the tie is biodegradable.

20. A method of coupling a stent to a blood vessel, the method comprising:
deploying a stent in a target blood vessel segment, the stent having a non-circular biased shape;
forcing the stent to a more-circular shape compared to the non-circular biased shape using a cylindrical scaffold positioned within a lumen of the stent and expanded to an expanded configuration wherein an outer diameter of the cylindrical scaffold is substantially equal to an inner diameter of the stent;
maintaining the cylindrical scaffold in the expanded configuration within the lumen of the stent for a period of time sufficient to allow tissue overgrowth to couple the stent to a wall of the target blood vessel segment; and
removing force of the cylindrical scaffold from the inner diameter of the stent to allow the stent to reshape the blood vessel segment to a non-circular shape.
